(19) United States
(12) Patent Application Publication
(10) Pub. No.: US 2021/0370288 A1
(43) Pub. Date: Dec. 2, 2021

(54) PURGE SWAB

(71) Applicant: AnuCell Biosystems Limited, Limerick (IE)

(72) Inventors: John Daly, Tipperary (IE); Kieran Curran, Limerick (IE); Macdara Glynn, Galway (IE); David Merrigan, Clare (IE); Jason Ryan, Tipperary (IE)

(21) Appl. No.: 17/332,677

(22) Filed: May 27, 2021

Related U.S. Application Data
(60) Provisional application No. 63/030,741, filed on May 27, 2020.

(51) Int. Cl. B01L 3/00; G01N 1/02

(52) U.S. Cl. CPC B01L 3/5029; A61F 13/38; B01L 3/50825; G01N 1/02

(57) ABSTRACT

Described herein are purge swab apparatuses, purge swabs kits, and methods for collecting a sample using a purge swab. In various embodiments, a purge swab includes an elongated body having a proximal end, a distal end, and a length therebetween. The purge swab further includes a swab head having a proximal end and a distal end wherein the proximal end of the swab head is attached to the distal end of the elongated body and the swab head includes a plurality of openings. The purge swab optionally includes a lumen extending from the proximal or distal end of the elongated body into at least a portion of the swab head, and the lumen is in fluidic communication with the plurality of openings.

### CROSS-REFERENCE TO RELATED APPLICATION

This application claims the benefit of U.S. Provisional Patent Application No. 63/030,741, filed on May 27, 2020, which is hereby incorporated by reference in its entirety.

### BACKGROUND

Embodiments of the present disclosure generally relate to swabs for biological sample collection. Swabs may be generally described as a wad of absorbent material (e.g., cotton) wound around one end of a small stick (e.g., paper, plastic, or wood) and may be used for sample collection in many types of biological tests. For example, swabs may be used for DNA, RNA, bacterial, viral, blood, and/or fecal testing. However, commercially-available swab kits do not fully transfer the collected sample during extraction, which may affect accuracy and precision of the resulting test. Accordingly, a new swab is needed that recovers more of the collected sample for biological processing and analysis.

### SUMMARY OF THE DISCLOSED SUBJECT MATTER

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, the disclosed subject matter includes an apparatus including an elongated body having a proximal end, a distal end, and a length therebetween. The apparatus further includes a swab head having a proximal end and a distal end, and the proximal end of the swab head is attached to the distal end of the elongated body. The swab head includes a plurality of openings. The apparatus further includes a lumen extending from the proximal end of the elongated body into at least a portion of the swab head. The lumen is in fluidic communication with the plurality of openings.

In various embodiments, an apparatus includes an elongated body having a proximal end, a distal end, and a length therebetween along a longitudinal axis. The apparatus further includes a swab head having a proximal end and a distal end, and the proximal end of the swab head is attached to the distal end of the elongated body. The swab head includes a plurality of openings. The apparatus further includes a bore extending along the longitudinal axis from the distal end of the swab head at least partially into the swab head. The bore is in fluidic communication with the plurality of openings, and the bore is configured to engage a pin of a container when the swab is inserted into the container thereby forcing fluid through the plurality of openings.

In various embodiments, an apparatus includes an elongated body having a proximal end, a distal end, and a length therebetween. The apparatus further includes a swab head having a proximal end and a distal end, and the proximal end of the swab head is attached to the distal end of the elongated body. The swab head comprises a plurality of surface features (e.g., grooves, bumps, scoring, etc.) on an outer surface of the swab head.

It is to be understood that both the foregoing general description and the following detailed description are exemplary and are intended to provide further explanation of the disclosed subject matter claimed.

The accompanying drawings, which are incorporated in and constitute part of this specification, are included to illustrate and provide a further understanding of the method and system of the disclosed subject matter. Together with the description, the drawings serve to explain the principles of the disclosed subject matter.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIG. 1 illustrates sample collection and recovery for a standard swab and a purge swab according to embodiments of the present disclosure.

FIG. 2A illustrates an exemplary purge swab according to embodiments of the present disclosure. FIGS. 2B-2D illustrate exemplary swab heads according to embodiments of the present disclosure.

FIGS. 3A-3B illustrate exemplary purge swab heads having indentures according to embodiments of the present disclosure. FIG. 3C illustrates a cross section of the swab head according to embodiments of the present disclosure.

FIGS. 4A-4B illustrate an exemplary pin-purge system according to embodiments of the present disclosure.

FIGS. 5A-5E illustrate a sample collection method and recovery using a pin purge swab according to embodiments of the present disclosure.

FIG. 6 illustrates a cross section of an exemplary pin-purge system according to embodiments of the present disclosure.

FIG. 7A illustrates a top-down view of the inside of a pin-purge system according to embodiments of the present disclosure. FIG. 7B illustrates a cross section of the pin-purge system according to embodiments of the present disclosure.

FIGS. 8A-8B illustrate an exemplary abrasion-purge system according to embodiments of the present disclosure.

FIGS. 9A-9D illustrate steps for collection and recovery using an abrasion-purge swab according to embodiments of the present disclosure.

FIG. 10A illustrates an exemplary spiral groove purge system according to embodiments of the present disclosure. FIG. 10B illustrates a cross section of the spiral groove purge system according to embodiments of the present disclosure.

FIGS. 11A-11B illustrate a cross-sectional rendering of a pin-purge system having a helical groove according to embodiments of the present disclosure. FIGS. 11C-11D illustrate a line drawing of a pin-purge system having a helical groove according to embodiments of the present disclosure.

FIGS. 12A-12D illustrate various embodiments of swab heads according to embodiments of the present disclosure.

FIGS. 13A-13B illustrate a piston-powered purge swab system according to embodiments of the present disclosure.

FIG. 14 illustrates a gas-powered purge swab system according to embodiments of the present disclosure.

FIGS. 15A-15C illustrate various charts related to purge swabs according to embodiments of the present disclosure.

FIG. 16 illustrates a chart of sensitivity fold change according to embodiments of the present disclosure.

FIGS. 17A-17G illustrate a cap dropper purge swab according to embodiments of the present disclosure.

FIGS. 18A-18J illustrate a gas-powered purge swab according to embodiments of the present disclosure.

FIGS. 19A-19G illustrate a syringe purge swab according to embodiments of the present disclosure.

### DETAILED DESCRIPTION

In various embodiments, a purge swab includes an elongated body having a proximal end and a distal end, and a swab head at the distal end of the body. In various embodiments, the purge swab may be used to collect a biological sample from a target (e.g., human, animal, etc.). For example, the purge swab may be inserted into a nasal passageway of a human such that the swab head contacts nasal secretions that are collected thereon. In various embodiments, the swab head is removed from the nasal passageway, inserted into a container (e.g., a tubular specimen container), and purged to more efficiently dislodge the collected sample from the swab head and/or transfer (e.g., disperse) the collected sample into a collection fluid. In various embodiments, purging of the swab head may be achieved in any suitable way to thereby improve the transfer of collected sample material into the container (e.g., into the collection fluid). In various embodiments, the elongated body has an axial lumen along its length. In various embodiments, because the purge swab more efficiently transfers collected sample from the swab head and into the container, the purge swabs described herein may reduce the amount of reagents required for downstream processing of the sample. In various embodiments, the purge swabs described herein may reduce the number of steps required for downstream processing.

In various embodiments, a purge swab and container may handle sample collection to sample recovery. In various embodiments, sample recovery may be based on a purging mechanism. In various embodiments, sample recovery may be based on an abrasive pumping mechanism. In various embodiments, purging is the forcing of liquid through one or more (e.g., a plurality of) apertures at the collection area (e.g., the swab head) to recover the specimen sample. In various embodiments, the purge swab includes an aperture array at the swab head. In various embodiments, although the aperture array may be designed differently with respect to the purge technique, the objective of the array is to provide jets of liquid adjacent to and striking zones of high sample collection in the swab head. In various embodiments, this is achieved by utilizing the variation in aperture shape to negate the pressure drop associated to the system and to maintain force of the purge across the swab head. In various embodiments, the aperture(s) may be square, rectangular, circular, ovular, or any other suitable shape.

In various embodiments, one category of purge swab may be a shaft stored purge (SSP). In various embodiments, another category of purge swab may be a collection tube stored purge (CTSP). In various embodiments, yet another category of purge swab may be an external fluid purge.

In various embodiments, a fluid (e.g., buffer solution, qPCR solution, etc.) may be contained within at least a portion (e.g., a proximal portion) of the lumen. In various embodiments, the fluid may be forced out of the lumen and out of a plurality of openings in the swab head. In various embodiments, the resulting flow through the openings in the swab head may improve the transfer of collected sample material from the swab head into the container.

In various embodiments, after the purge swab is inserted into the container (and the swab head is seated within the seating portion), at least a portion of the elongated body may be removed (e.g., broken off) to thereby allow the cap to be affixed (e.g., screwed on) to the container. In various embodiments, the elongated body of the purge swab may be marked (e.g., scored) at a predetermined location so that a user knows where to break off the elongate body after sample collection.

In various embodiments, a SSP swab may be implemented using a pumping mechanism (e.g., a plunger). In various embodiments, the purge swab may be capped (e.g., sealed) after use within the associated collection tube. In various embodiments, a purge liquid may be stored within a lumen of the body of the purge swab. In various embodiments, the purge liquid may be stored between a frangible layer (e.g., film, foil, sheath, etc.) and the pumping mechanism (e.g., a plunger). In various embodiments, the purge swab may be manually pumped by the user (after the purge swab is capped inside the collection tube) to thereby force the purge liquid out of the swab head. In various embodiments, the cap may include a purge mechanism (e.g., a piston) configured to engage and force the pumping mechanism (e.g., plunger) in the purge swab distally into the body of the purge swab thereby forcing the liquid optionally past the frangible layer and through one or more apertures in the swab head, thus transferring material captured by the swab head into the collection tube. In various embodiments, the piston may be forced down a lumen of the shaft, thereby ejecting a purge liquid through one or more apertures in the swab tip. In various embodiments, the piston may reach the full length of the swab tip, thus providing pressure to push purge liquid through the last apertures which would have less pressure in other designs. In various embodiments, the piston mechanism may be integrated into the cap of the collection tube. In various embodiments, the piston may be a separate component from the collection tube, and may be inserted into the cap after the cap has been attached (e.g., screwed onto) the collection tube.

In various embodiments, rather than using a piston to force the purge liquid through the lumen of the purge swab, a compressed gas (e.g., air or other inert and non-reactive gas such as CO₂ or nitrogen) may be used to force the purge liquid through the lumen and out the one or more apertures in the swab head. In various embodiments, the cap may contain a compressed gas configured to force the purge liquid through the swab head. In various embodiments, forcing the purge liquid through the lumen may be triggered by screwing the collection tube cap onto the collection tube (e.g., after the cap has been fully screwed on the collection tube). In various embodiments, the cap may include a gas port configured to receive a compressed gas (e.g., CO₂) cartridge configured to provide compressed gas and force the purge liquid through the swab head.

In various embodiments, a cap dropper may be used to provide purge liquid to the lumen of the purge swab. In various embodiments, the cap dropper includes a cap having a rubber bulb and a tube extending from the bulb and through the cap (e.g., a Pasteur pipette). In various embodiments, a syringe may be used to provide purge liquid to the system via the cap. In various embodiments, where the purge liquid is contained within the cap or the swab itself, a locking system may be used to ensure the purge liquid cannot be activated prematurely. In various embodiments, the locking system may include a frangible piece of material (e.g., plastic) that requires a predetermined amount of force to break. In various embodiments, the locking system may include a pin (e.g., split pin, cotter pin, etc.). Premature purging could result in redundancy of the swab, loss of the sample, or jetting of the liquid into the patient during sample collection. In various embodiments, when a locking system is included, purging may only occur after capping the purge swab into its associated collection tube.

In various embodiments, any suitable locking system may be used to ensure purging cannot be activated prematurely. In various embodiments, for a syringe type purge system, a clip may be positioned between a thumb pad of the plunger and the syringe body. In various embodiments, the clip may be made of any suitable material (e.g., plastic, metal). In various embodiments, the clip may be any suitable color, for example, a bright color (e.g., red) to direct attention to the clip. In various embodiments, for a dropper-type purge system, an additional screw-on cap may protect the rubber bulb of the dropper. In various embodiments, the cap may be a screw-on cap. In various embodiments, the cap may be any suitable color, e.g., red. In various embodiments, for a piston-type purge system, a circlip (C-clip) may be placed close to a distal end of the piston (e.g., over a circumferential groove), necessitating removal of the circlip before the purge step. In various embodiments, for a compressed gas purge system, the compressed gas canister may include a frangible seal that is pierced when the canister is screwed into a receptacle. In various embodiments, the receptacle may be formed partly by the tube and partly by the tube cap, thereby forming an interlock to only allow canister insertion to a completed tube/cap assembly. In various embodiments, the cap and/or tube may include a metering orifice. In various embodiments the cap and/or tube may include a filtered vent arrangement to mitigate over-pressurization of the tube assembly.

In various embodiments, purge liquid may be stored within a frangible vessel (e.g., a disc). In various embodiments, purge liquid may be stored as a semi-solid matrix. In various embodiments, purge liquid may be stored within a channel or reservoir structure within the purge swap and/or cap. In various embodiments, if liquid is stored in the swab, the purge swap may include an upper seal and/or lower seal to maintain isolation from the ambient environment. In various embodiments, the lower seal may separate the liquid from the swab material. In various embodiments, the lower seal may be a pressure frangible material (e.g., plastic, foil, sheet, etc.). In various embodiments, the upper seal may be a frangible material (e.g., plastic, foil, sheet, etc.). In various embodiments, the upper seal may be a deformable membrane configured to maintain a barrier with the ambient environment even after activation.

In various embodiments, a fluid (e.g., buffer solution, qPCR solution, etc.) may be forced into a fluidic connection in the proximal end of the body such that the fluid flows through the lumen and out of a plurality of openings in the swab head. In various embodiments, the resulting fluid flow through the openings in the swab head improves the transfer of collected sample material from the swab head into the fluid.

In various embodiments, a CTSP swab may use a conventional swab shaft (e.g., a solid shaft). In various embodiments, the purge liquid may be stored within the collection tube. In various embodiments, the collection tube may include a molded-in pin feature at the bottom of the tube. In various embodiments, the collection tube may include one or more (e.g., two, three, four, etc.) guidance fins on the inner diameter of the tube configured to guide pumping of the swab up and down during the purging step. In various embodiments, a user may pump the swab head up and down one or more times to draw the purge liquid in and force the purge liquid out of or around the swab head. In various embodiments, larger ports towards the distal end may increase distal flushing in the initial part of the downward stroke. In various embodiments, smaller ports at the proximal end may be flushed when the pin acts like a piston closing ports during the down stroke.

In various embodiments, the purge swab may include a one-way valve that allows fluid flow in a single direction. For example, a one-way valve may only allow purge liquid (e.g., VTM) to flow out of the swab head. In various embodiments, a two-way valve may be incorporated into the swab to provide recirculation of VTM from the distal end of the swab tip and through the apertures along the tip.

In various embodiments, the purge system may use the walls of the collection tube to engage with the swab tip at any point during insertion of the swab head into the collection tube. In various embodiments, repeated pumping of the swab head into the collection tube may provide abrasive contact between an engagement structure within the tube (e.g., a a coarse inner surface of the tube and/or structure(s) configured to modify, e.g., deform, the surface of an object passing therethrough). For example, an inner surface of the collection tube may include one or more circumferential ribs and/or vertical fins configured to engage the swab head as the swab head is inserted into the collection tube.

In various embodiments, the swab head may be molded from a polymer template. In various embodiments, two or more polymer templates may be used for different portions of the purge swab, and the portions of the purge swab may be assembled together. In various embodiments, the polymer template of the purge swab may be designed to incorporate rough textures, which act as accumulation zones for the sample. In various embodiments, sample collection with swabs often requires a level of disruption to the zone of collection (e.g., the nasopharyngeal passage). In various embodiments, disruption may be performed by scratching, scraping, or rolling the swab along a surface of the collection zone. In various embodiments, the purge swab may be designed to collect (e.g., accumulate) sample using any suitable disruption technique.

In various embodiments, the outer surface of the swab head may include a double helix collection structure. In various embodiments, the collection structure may include sharp edges. In various embodiments, the collection structure may include rough textures to thereby provide a course surface to improve sample collection. In various embodiments, rolling the swab head in one or both the clockwise and anti-clockwise directions is possible because of the bidirectionality of the collection structure.

In various embodiments, the collection zones may be symmetrical 90-degree cut outs from the swab tip which provide the accumulation zones for the sample. In various embodiments, the swab tip may be cylindrical in shape. In various embodiments, zones and/or textures may be added to increase surface roughness on the tip, thus increasing the collection ability of the swab head. In various embodiments, the surface of the swab head may

In various embodiments, the purge swab may be designed to be injection molded. In various embodiments, the purge swab may be designed to be over-molded. In various embodiments, the particular manufacturing method may depend on the complexity of the design and the capabilities of the molding process. In various embodiments, the narrow-hollowed shaft may be manufactured using over-molding. In various embodiments, the narrow-hollowed shaft may be manufactured using injection molding.

In various embodiments, the purge swab may be designed for naso-pharyngeal sample (e.g., mucus) collection. In various embodiments, the design of the purge swab may be similar to that of commercially-available oral swabs. In various embodiments, the purge swab head design may be altered to suit the desired application, as can the shaft.

In various embodiments, the swabs described herein may be used in applications such as: nasal, throat, vaginal, groin, armpit, rectal, wound, buccal, faeces, urethral, pediatric, neonatal, endo-esocervial, endocervical and mid turbinate. With the flexibility in the design of the invention regarding the tip structure, the purge system and the volume delivered for collection; all of these applications are feasibly catered for. In various embodiments, a purge swab (e.g., a syringe embodiment and with a syringe attached via a luer adapter) may be used for other surgical and non-surgical wash/purge/drain/irrigate operations, e.g., saline flush of vessels or structures during micro surgery or ear wax removal (e.g., a hot water purge).

In various embodiments, the purge swab may be used in combination with a receiving vessel to stabilize the sample. In various embodiments, the sample may be processed (e.g., with a lysis buffer) following sample collection. In various embodiments, the sample may be further processed, for example, via a Torus device (as disclosed in U.S. Pat. No. 11,000,780, which is hereby incorporated by reference in its entirety) or a microfluidic card using capillary motion to thereby generate a stable sample prior to isolation, diagnostic testing, and/or molecular testing.

In various embodiments, the purge liquid may include any suitable fluid for transferring a sample disposed on the swab head (after sample collection) into the collection container. In various embodiments, the purge liquid may include water. In various embodiments, the purge liquid may include Viral Transport Media (VTM). In various embodiments, the purge liquid may include a buffered saline solution. In various embodiments, the purge liquid may be sterile. In various embodiments, the purge liquid may include DNase and/or RNase-free water or buffer for embodiments testing for nucleic acid. In various embodiments, the purge liquid may include sterile culture media. In various embodiments, the purge liquid may include bacterial transfer fluid for recovery of contaminating bacteria from a location. In various embodiments, the purge liquid may include one or more chemical indicator for embodiments where presence of an analyte can be qualitatively detected by addition of a single chemistry (i.e., similar to Clean-Trace from 3M). In various embodiments, the purge liquid may include a first reagent of a dual reagent test such as the Kastle-Meyer blood detection test where the phenolphthalein is loaded in the swab and the H₂O₂ is added by a dropper. In various embodiments, the swab may dispense two liquids such that each liquid is released in series. In various embodiments, the purge liquid may be substituted with a gas, e.g., air. In various embodiments, the purge liquid may include one or more mineral oils having, e.g., silicon or fully/partially fluorinated compositions.

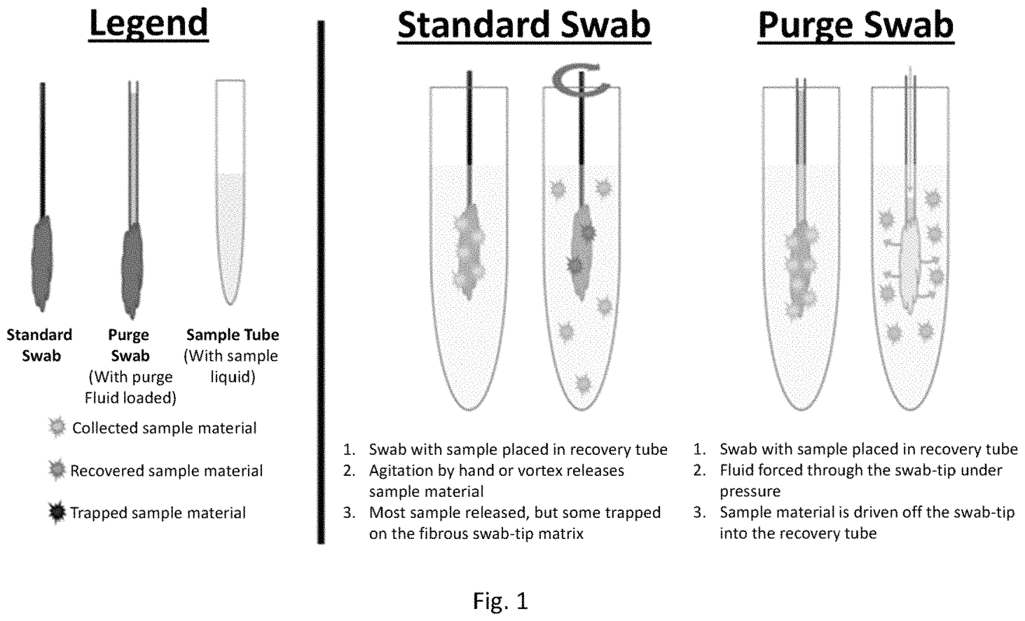

FIG. 1 illustrates sample collection and recovery for a standard swab and a purge swab. As shown in FIG. 1, the standard swab fails to complete recovery of the collected biological material because embedded sample remains hard to reach (e.g., adhered to the fibrous swab, embedded within) and despite agitation, is not recovered into the sample liquid. In various embodiments, a purge swab utilizes a purging liquid to maximize recovery, flushing the sample from the inside out. As shown in FIG. 1, in contrast to the standard swab, a purge swab using a purge liquid recovers more collected biological sample material on the swab. In various embodiments, the purge swab may recover over 50% of collected material. In various embodiments, the purge swab may recover over 60% of collected material. In various embodiments, the purge swab may recover over 70% of collected material. In various embodiments, the purge swab may recover over 80% of collected material. In various embodiments, the purge swab may recover over 90% of collected material. In various embodiments, the purge swab may recover over 95% of collected material.

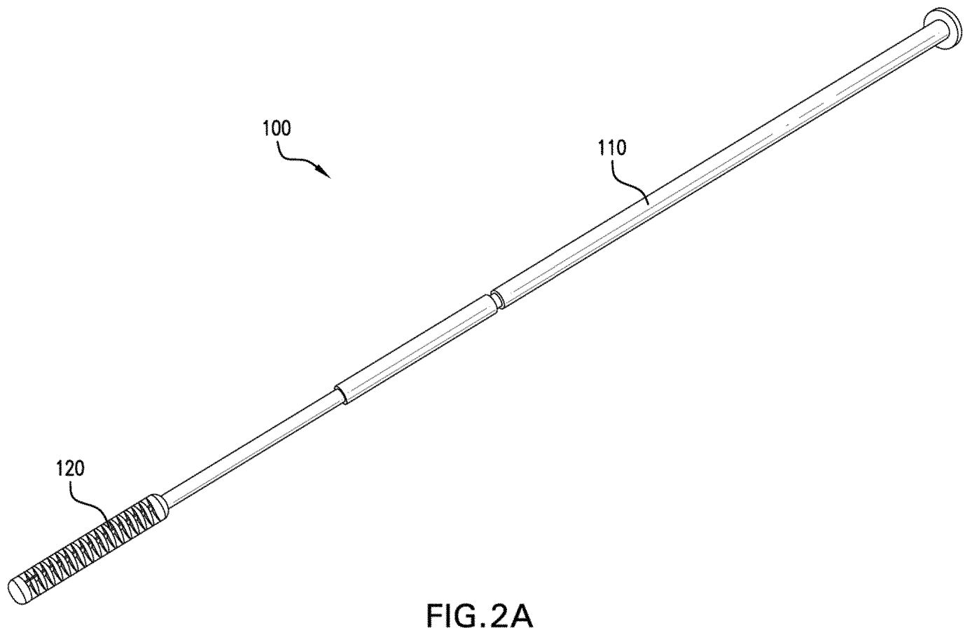

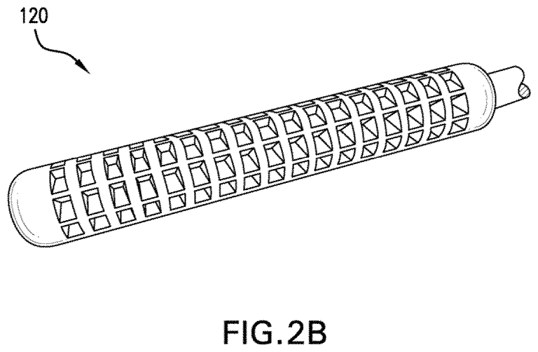

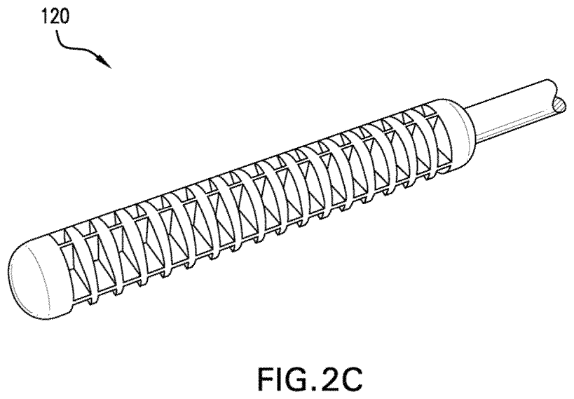

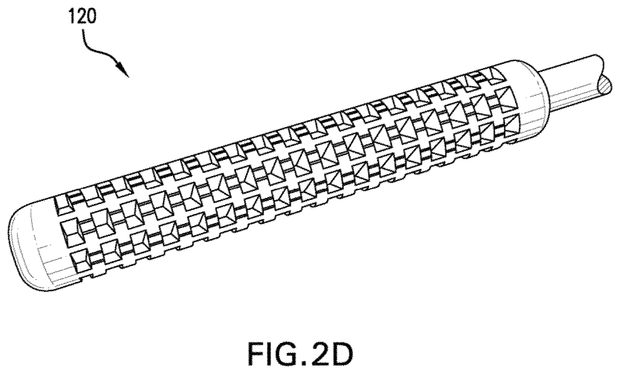

FIG. 2A illustrates an exemplary purge swab. The purge swab 100 includes an elongated body 110 and a swab head 120. In various embodiments, the swab is injection molded with a thin shaft core to produce the hollowed region for purging the collected sample. The swab heads vary in complexity based on the required surface roughness for the application. FIGS. 2B-2D illustrate exemplary swab heads. As shown in FIGS. 2B-2D, the swab head 120 may include a plurality of concentric rings or discs having a predetermined spacing therebetween, or stacked. In various embodiments, the swab head 120 may further include one or more planar extrusions, as shown in FIG. 2B. In various embodiments, the swab head 120 may further include one or more radial extrusions extending radially outward from the lumen, as shown in FIG. 2C. In various embodiments, the swab head 120 may include scoring or indentures (e.g., grooves) to effectively roughen the surface of the swab head 120.

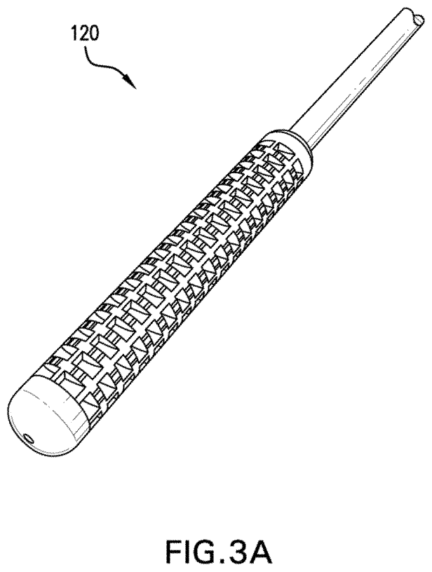

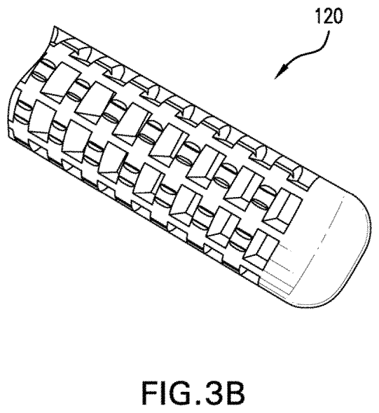

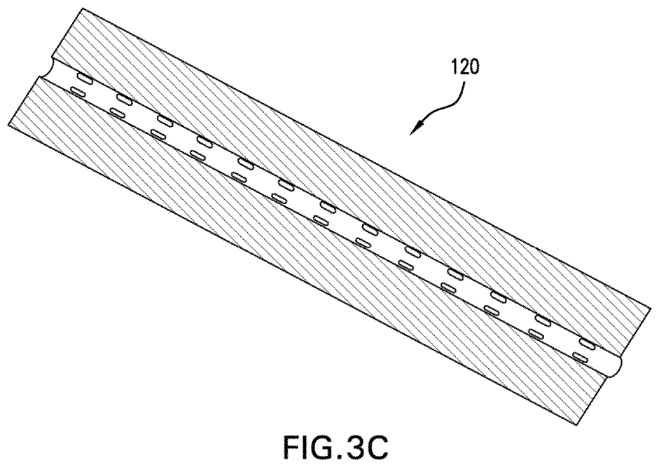

FIGS. 3A-3B illustrate exemplary purge swab heads having indentures to provide surface roughness. In various embodiments, the indentures serve as collection zones for accumulation of sample and are adjacent the openings for sample extraction. FIG. 3C illustrates a cross-section of a swab head 120 showing the hollow lumen and openings through which the purge liquid is jetted.

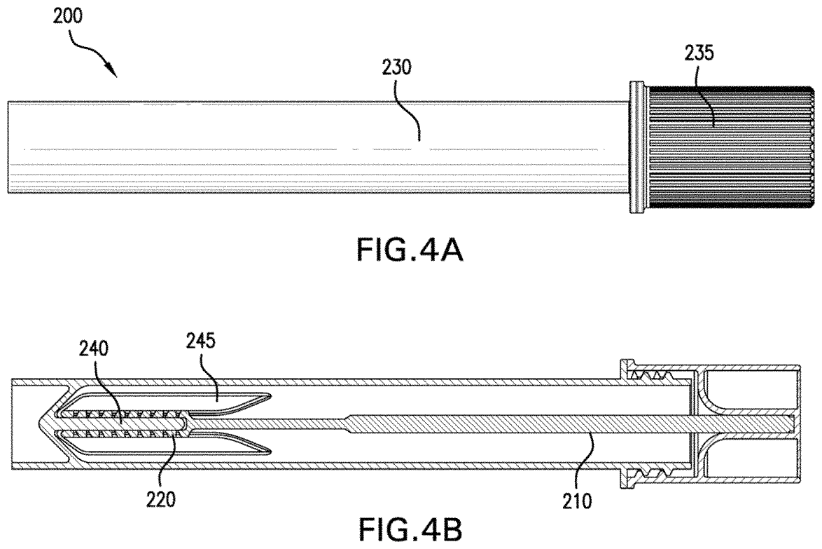

FIGS. 4A-4B illustrate an exemplary pin-purge system 200. In various embodiments, the container 230 includes a pin 240 at its distal end and the pin extends axially from the distal end towards the proximal opening of the container. The pin 240 engages a bore in the swab head 220 as the swab head 220 is inserted in the container 230 to thereby force fluid through the openings in the swab head 220. FIG. 4A illustrates a pin purge system 200; collection tube 230 and accompanying cap 235 with swab within. FIG. 4B illustrates the swab head 220 and body 210 centralized upon entry into the container (e.g., tube) 230 by fins 245 surrounding the pin 240 and extending radially inward. As the pin 240 enters the distal end of the swab head 220, displacement of the fluid at the bottom of the container 230 creates a pressurized purge which evacuates through the openings along the swab head 220, thus recovering the sample from the swab head 220. Additionally, the fins 245 can serve as a guide to orient the swab head 220 into alignment with a central longitudinal axis of the container 230. Thus, the purge swab can be inserted (manually and individually; and/or automatically and in bulk/batch processing into a plurality of containers) into the container 230 at any angle, and upon contacting the fins 245, the swab(s) is “straightened” or oriented to be oriented along the longitudinal axis of the container, thereby ensuring the distal end of the swab head 220 reaches the bottom of the container 230 reservoir (i.e., the seating portion).

In various embodiments, after the purge swab is inserted into the container 230, at least a portion of the elongated body 210 may be removed (e.g., broken off) to thereby allow the cap 235 to be affixed (e.g., screwed on) to the container 230.

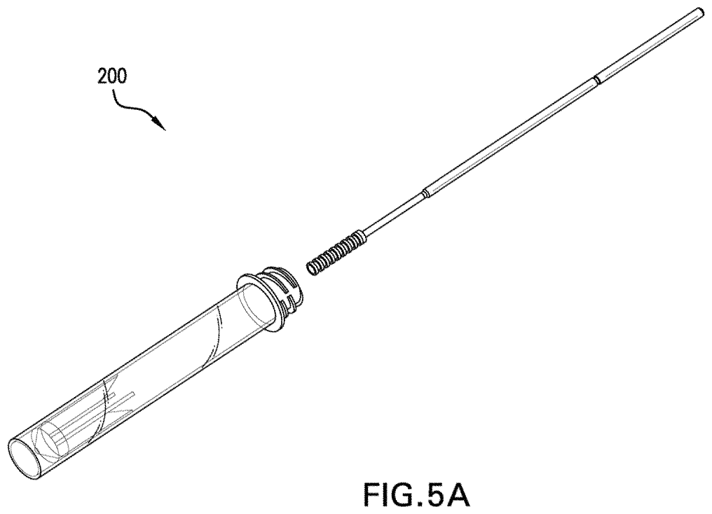

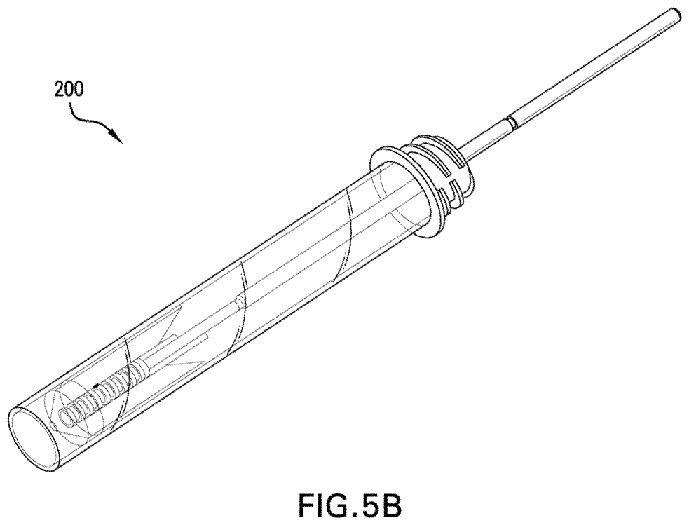

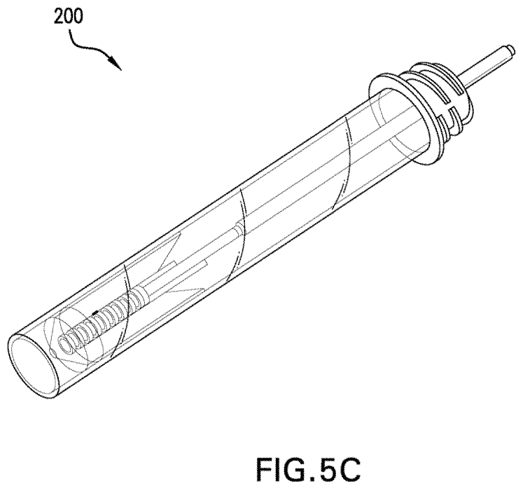

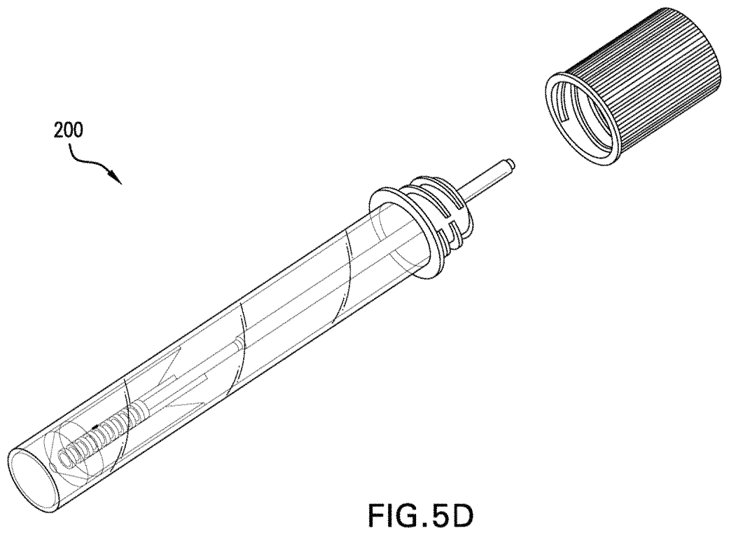

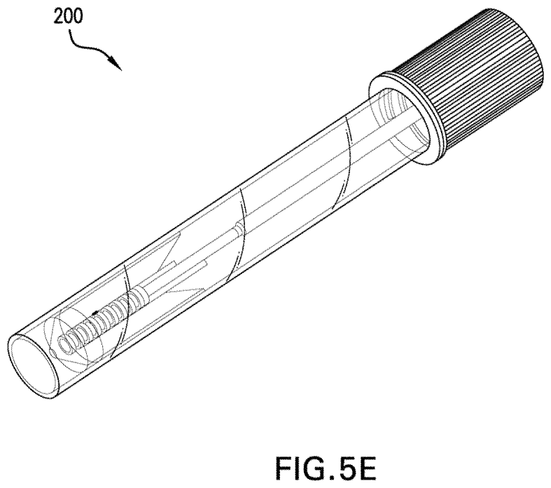

FIGS. 5A-5E illustrate a sample collection method and recovery using a pin-purge swab system 200. In a first step, shown in FIG. 5A, the swab is added to collection tube. In a second step, shown in FIG. 5B, the swab head is centralized by fins. In a third step, shown in FIG. 5C, the user plunges the swab up and down several times to pump fluid (e.g., VTM) from the swab central core to the outside via the arrayed radial orifices. In a fourth step, shown in FIG. 5D, the user breaks the swab at a breakpoint to remove the handling zone (i.e., the part touched by the user). In a fifth step, shown in FIG. 5E, the cap 235 of the container 230 is attached. In various embodiments, the cap 235 is also centralized by funnel feature, and interference fitted to swab to be pumped for purging and removed as one component upon receipt in lab. In another embodiment the cap can be interfaced with to allow for pumping and processing.

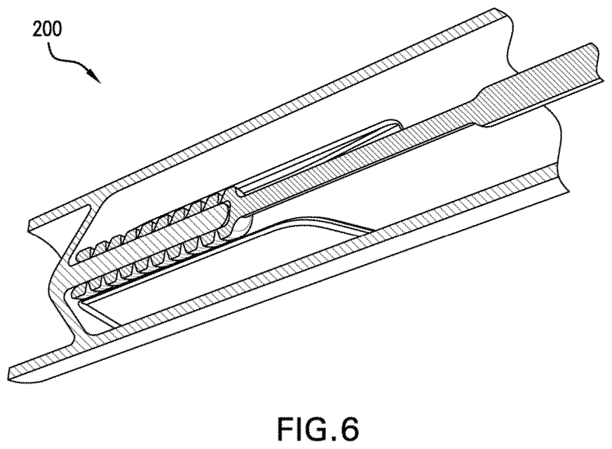

FIG. 6 illustrates a cross section of an exemplary pin-purge system 200 displaying fit between pin 240 and swab head 220.

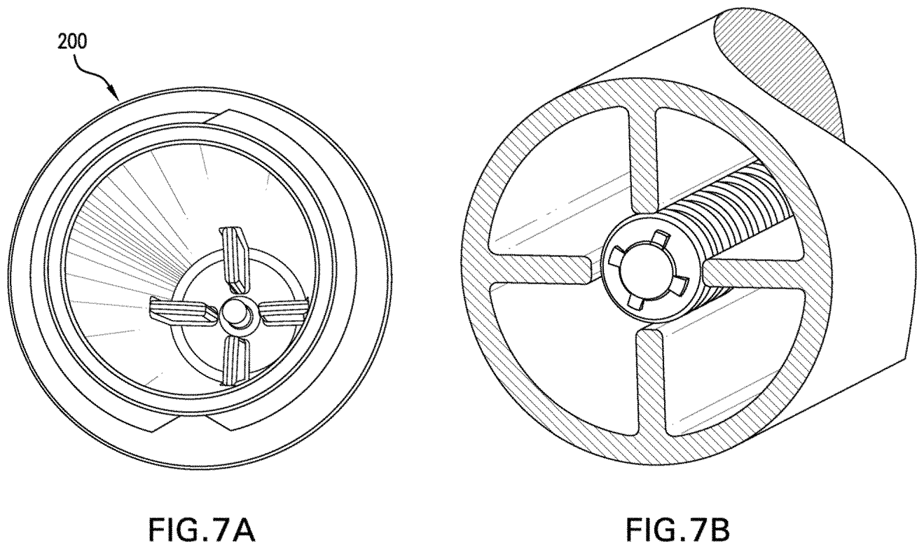

FIG. 7A illustrates a top-down view of the inside of a pin-purge system 200 and FIG. 7B illustrates a cross section of the pin-purge system 200. As shown in FIGS. 7A-7B, the container includes an axial pin 240 extending towards the proximal end (i.e., the opening) of the container and four guidance fins 245 disposed radially around the pin 240. In various embodiments, the fins may include a beveled or chamfered leading edge to center the swab head 220.

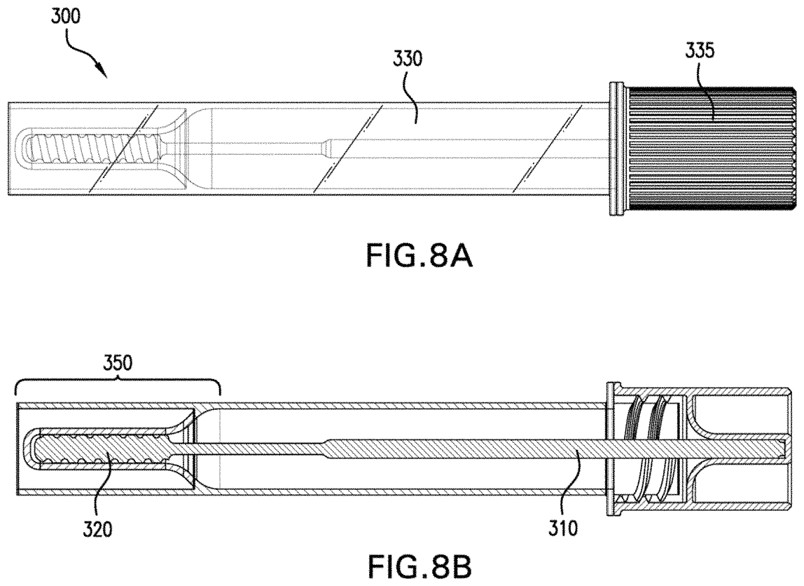

FIGS. 8A-8B illustrate an exemplary abrasion-purge system 300, including a collection tube 330 and accompanying cap 335 with swab having a swab head 320 and body 310. As shown in FIGS. 8A-8B, the swab is centralized upon entry into tube by a seating portion 350 having narrowing walls. In various embodiments, the seating portion 350 includes a smaller diameter than the diameter of the proximal opening of the container 330. Displacement of liquid as the swab head 320 is pushed into the seating portion 350 forces the liquid within to travel up the groove (e.g., threading) recovering the sample from these collection zones of the swab head. In various embodiments, the seating portion 350 may include one or more grooves (such as a helical groove or threading) on the inner surface adjacent the swab.

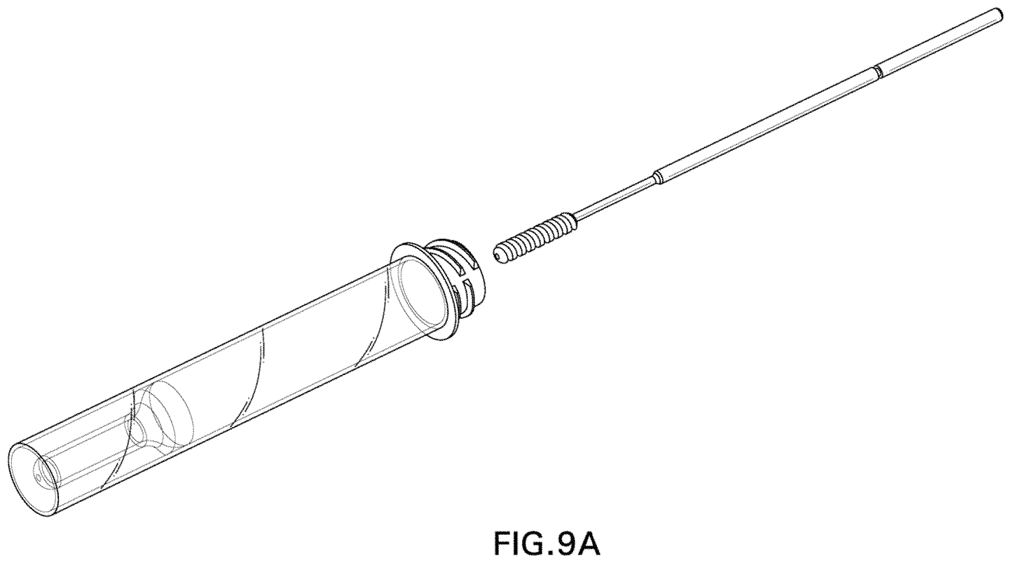

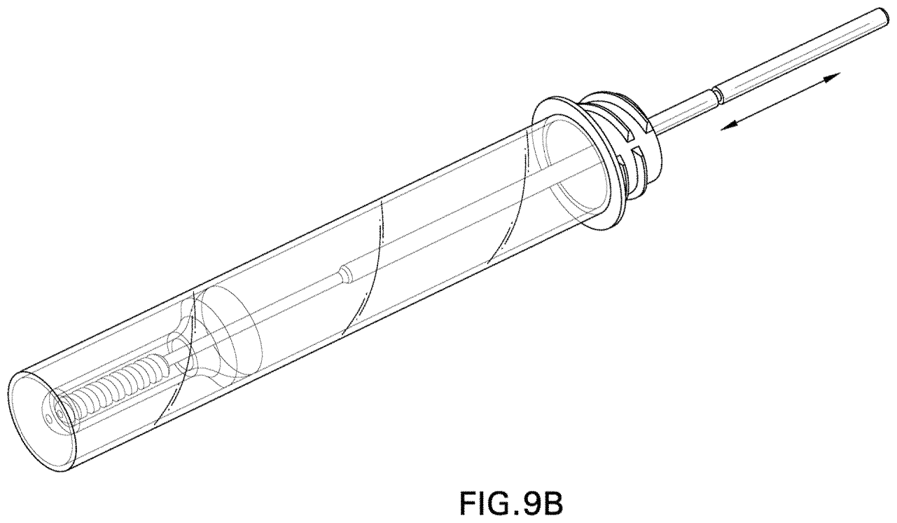

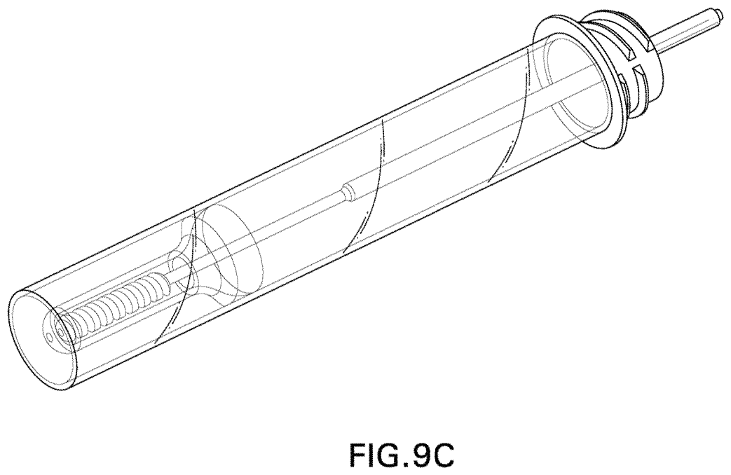

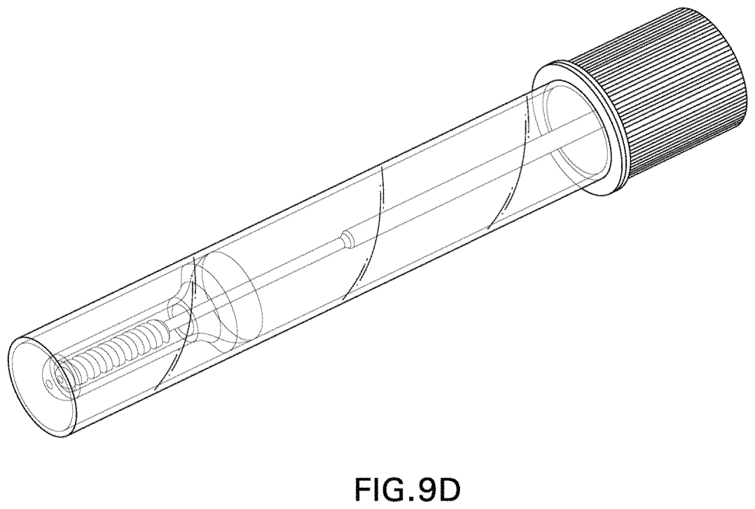

FIGS. 9A-9D illustrate steps for collection and recovery using an abrasion-purge swab. In a first step, shown in FIG. 9A, the swab is added to the container 330 and is centralized by the narrowing walls of the seating portion 350. In a second step, shown in FIG. 9B, the user plunges the swab up and down one or more times (e.g., several) to pump fluid (e.g., VTM) from the narrow chamber through the groove (e.g., helical) of the swab. In a third step, shown in FIG. 9C, the user breaks the swab at a breakpoint to remove the handling zone (i.e., the part touched by the user). In a fourth step, shown in FIG. 9D, the cap 335 of the container 330 is attached. In various embodiments, the cap 335 is also centralized by funnel feature, and interference fitted to swab to be pumped for purging and removed as one component upon receipt in lab.

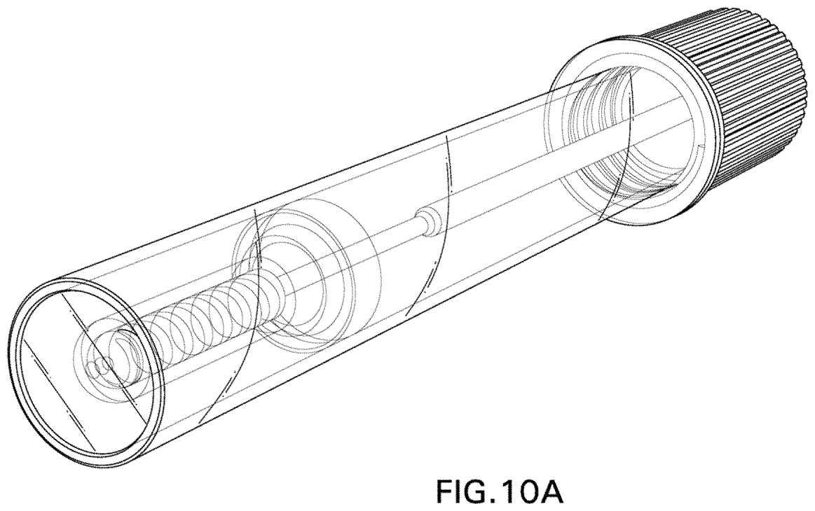

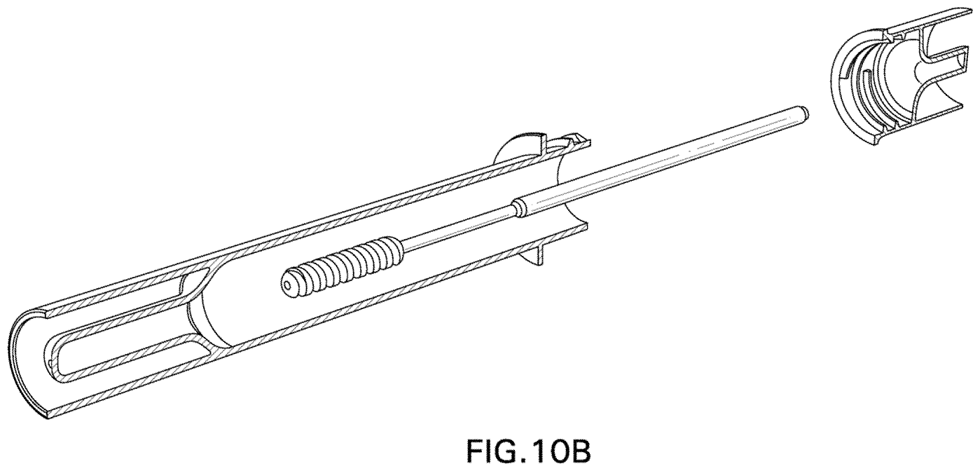

FIGS. 10A-10B show a helical groove feature creates (1) one or more (a plurality of) accumulation zones for the sample, and (2) a path through which the liquid, displaced upon addition of the swab to the tube, travels and recovers the sample into solution.

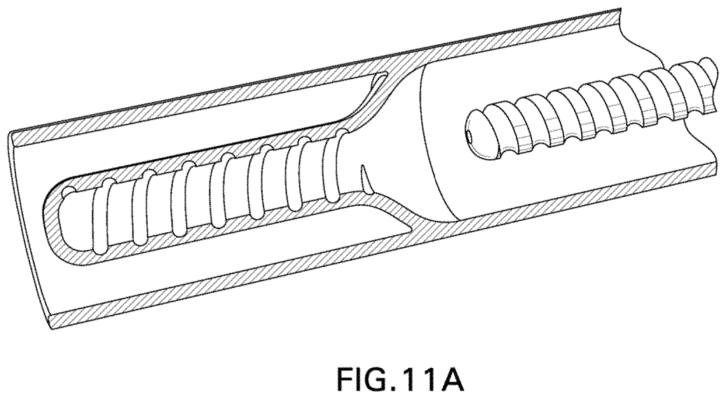

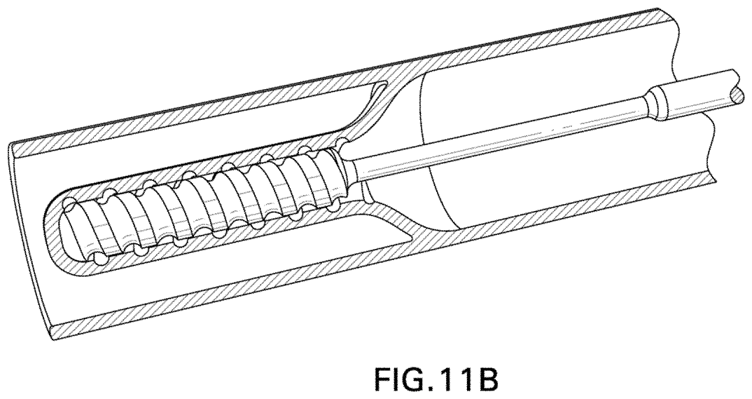

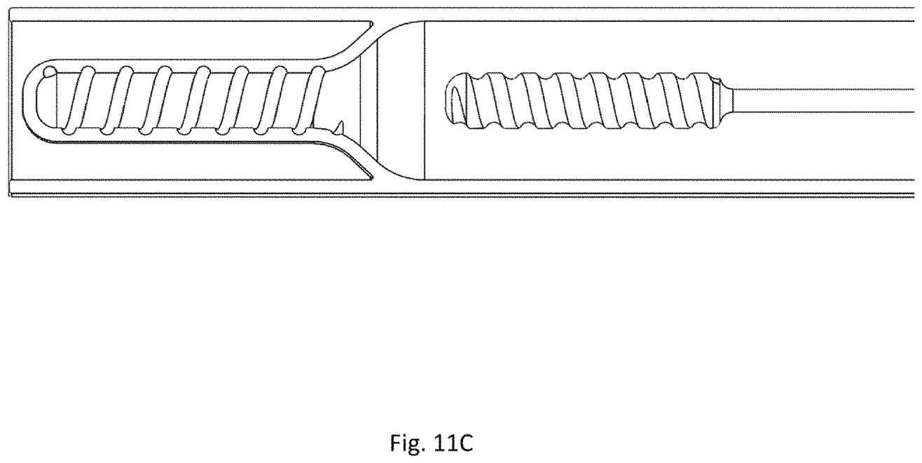

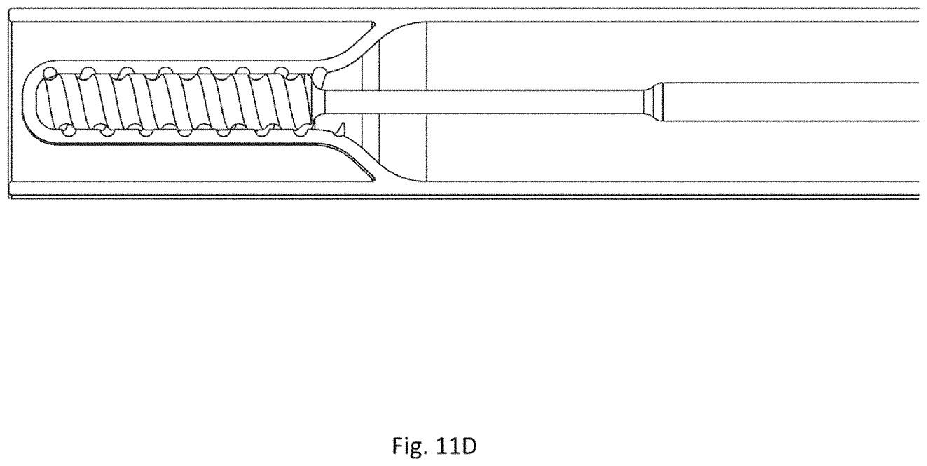

FIGS. 11A-11B illustrate a cross-sectional rendering of a pin-purge system having a helical groove according to embodiments of the present disclosure. FIGS. 11C-11D illustrate a line drawing of a pin-purge system having a helical groove according to embodiments of the present disclosure. In another embodiment, a cavity below the distal end may be used for the collection of fluid, e.g., sample.

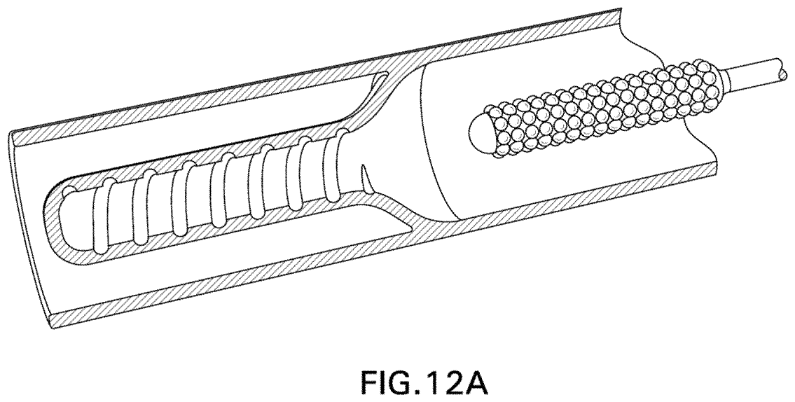

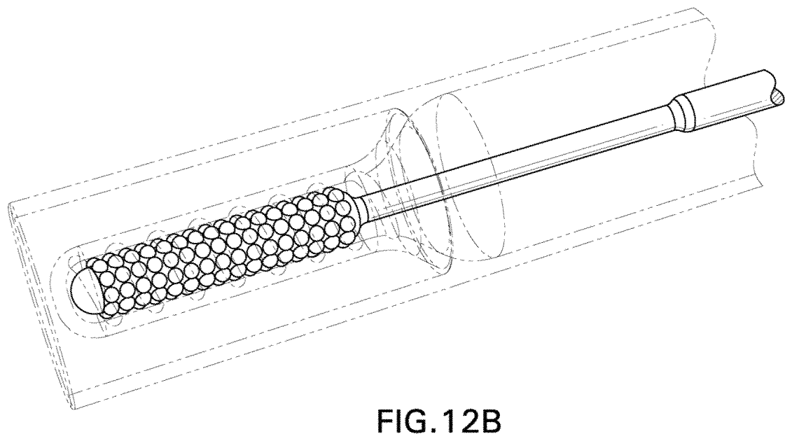

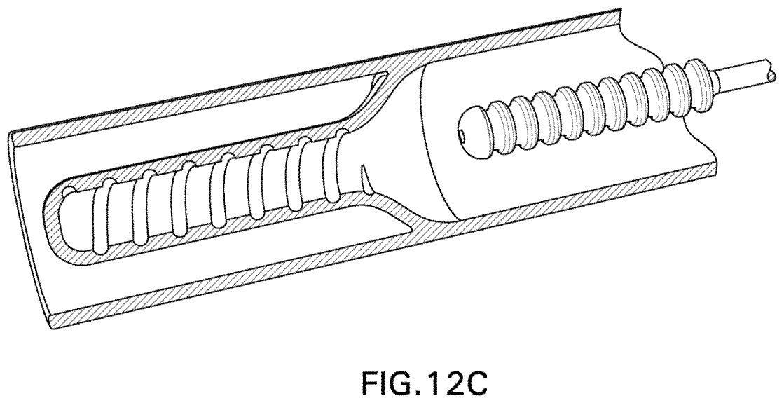

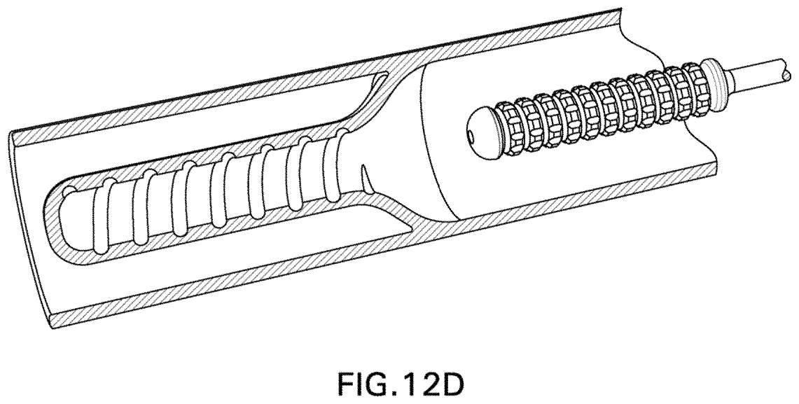

FIGS. 12A-12D illustrate various embodiments of swab heads according to embodiments of the present disclosure. In particular, the swab head in FIGS. 12A-12B includes a plurality of bumps configured to roughen the swab head surface and improve specimen collection. In FIG. 12C, the swab head includes a plurality of concentric grooves (e.g., in a honey-dipper shape). In various embodiments, the seating portion may include helical grooves. In various embodiments, the seating portion may include concentric grooves, similar to the grooves of the swab head. In FIG. 12D, the swab head includes a plurality of concentric grooves and the hills between the grooves include a plurality of longitudinal scoring or grooves to further roughen the swab head surface and improve specimen collection. In another embodiment, a cavity below the distal end may be used for the collection of fluid, e.g. sample. In another embodiment the interference between the swab and the container is sufficient to mechanically wipe the sample fluid from the swab.

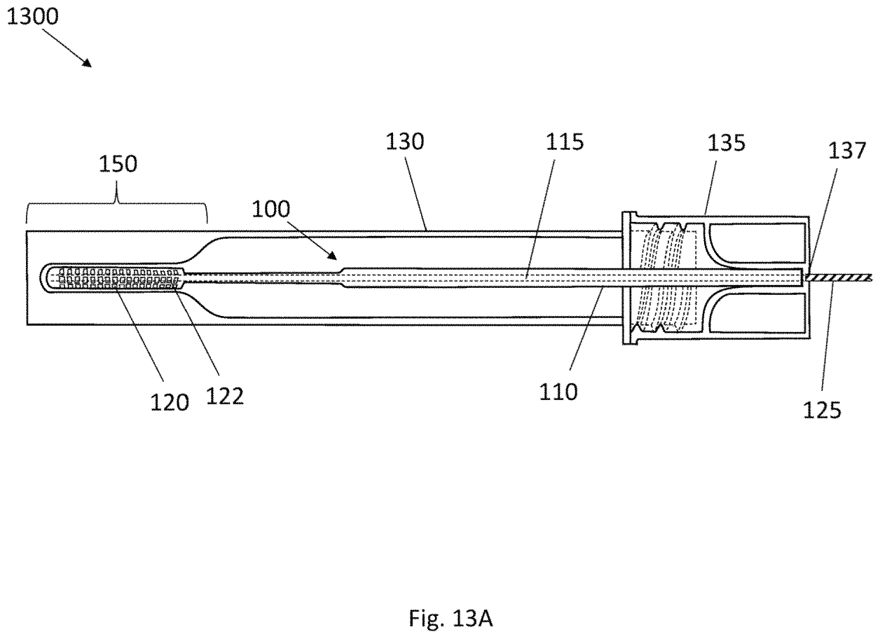

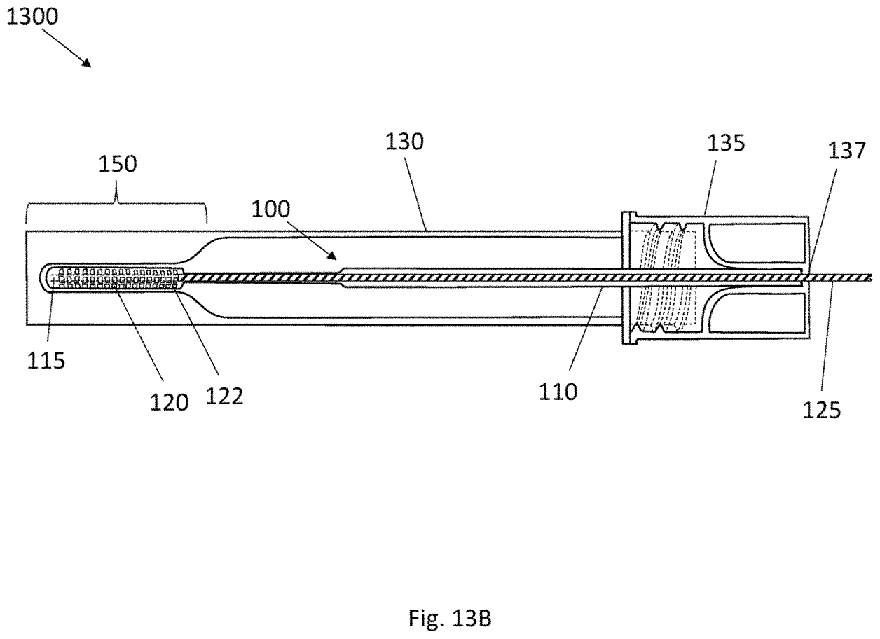

FIGS. 13A-13B illustrate a piston-powered purge swab system 1300. In particular, FIG. 13A illustrates a snapshot after the purge swab 100 has been inserted into the container 130 (e.g., collection tube) such that the swab head 120 is disposed within the seating portion 150 and substantially sealed with the cap 135. In various embodiments, a purge mechanism 125 (e.g., a piston) may be inserted into a port 137 in the cap 135 (that is in fluidic communication with the lumen 115) and into a lumen 115 of the elongate body 110 to thereby force purge liquid contained therein through the one or more apertures 122 in the swab head 120. In various embodiments, the purge mechanism 125 may be integrated into the cap 135 such that when the cap 135 is attached (e.g., fully screwed on), the purge mechanism 125 is activated (e.g., manually or automatically) to thereby force the purge liquid through the lumen 115 and out of the one or more apertures 122 of the swab head 120.

In various embodiments, the container 135 may include a predetermined amount of liquid in the container before the purge swab 100 is inserted. In various embodiments, the container 135 may be provided without liquid (i.e., dry).

FIG. 13B illustrates a snapshot after the purge mechanism 125 has been fully activated to thereby force the purge liquid through the lumen 115, into the swab head 120, out of the one or more aperture 122, and into the container 130. As the purge liquid is forced through the one or more aperture 122, the purge liquid may transfer at least a portion (e.g., substantially all) of the collected sample into the container 130, thereby increasing the yield of a single swab. In some embodiments the purge mechanism 125 can include a depth gauge (visible and/or audible) to indicate the distance inserted through the swab, thus allowing inspection and confirmation that a full range of purge mechanism insertion (and thus swab dispensing) has been completed.

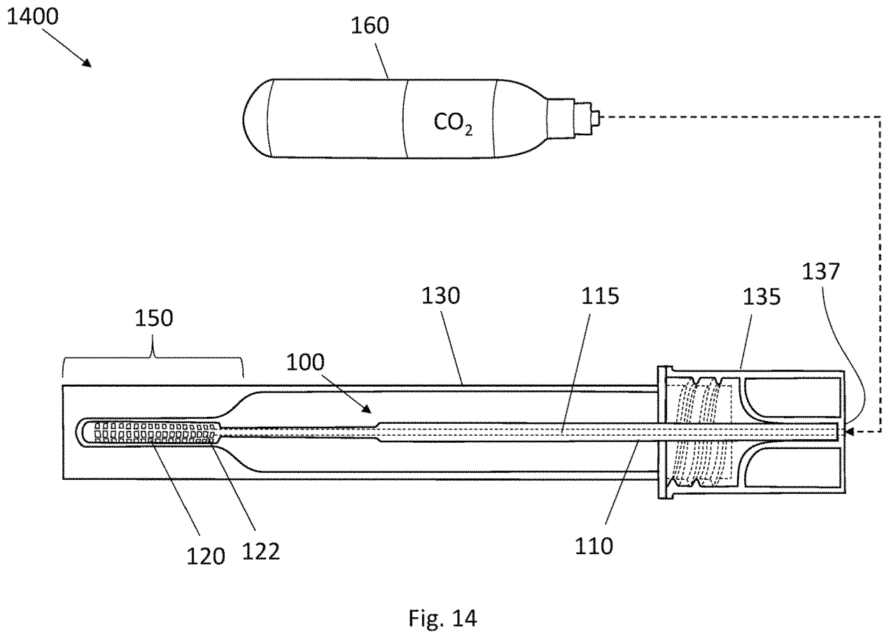

FIG. 14 illustrates a gas-powered purge swab system 1400. In particular, FIG. 13B illustrates a snapshot after the purge swab 100 has been inserted into the container 130 (e.g., collection tube) such that the swab head 120 is disposed within the seating portion 150 and substantially sealed with the cap 135. In various embodiments, a source of pressurized gas 160 (e.g., a pressurized gas cartridge, pressurized gas canister, etc.) may be provided with the purge swab system. In various embodiments, the source of pressurized gas 160 may be configured to attach to a port 137 in the cap 135 that is in fluidic communication with a lumen 115 of the elongate body 110. In various embodiments, the source of pressurized gas 160 may be configured to provide a predetermined amount of pressurized gas into the lumen 115 to thereby force purge liquid contained therein through the one or more apertures 122 in the swab head 120. In various embodiments, the source of pressurized gas 160 may be integrated into the cap 135 such that when the cap 135 is attached (e.g., fully screwed on), the source of pressurized gas 160 provides (e.g., manually or automatically) the gas to thereby force the purge liquid through the lumen 115 and out of the one or more apertures 122 of the swab head 120.

In various embodiments, the source of pressurized gas 160 may include pressurized air. In various embodiments, the source of pressurized gas 160 may include pressurized carbon dioxide. In various embodiments, the source of pressurized gas 160 may include any suitable pressurized gas that is configured to force the purge liquid through the lumen 115, into the swab head 120, out of the one or more aperture 122, and into the container 130.

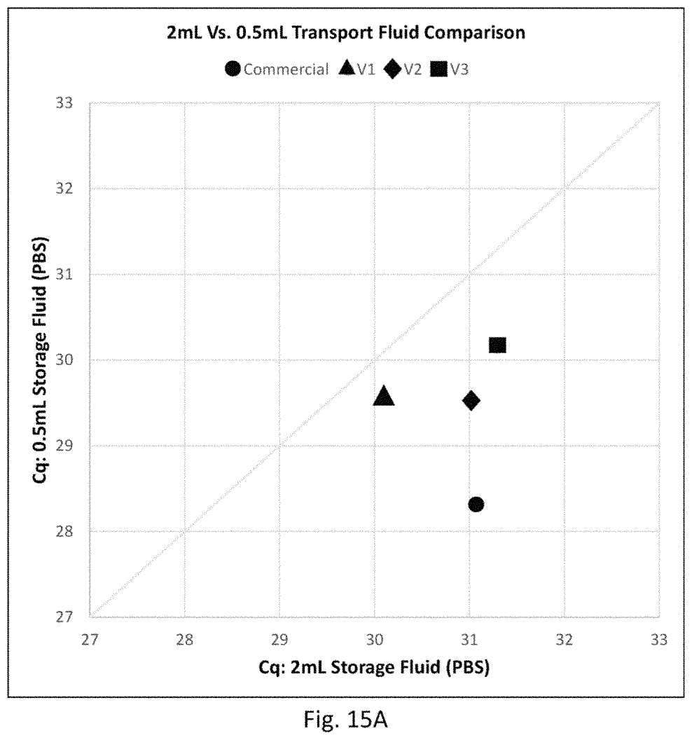

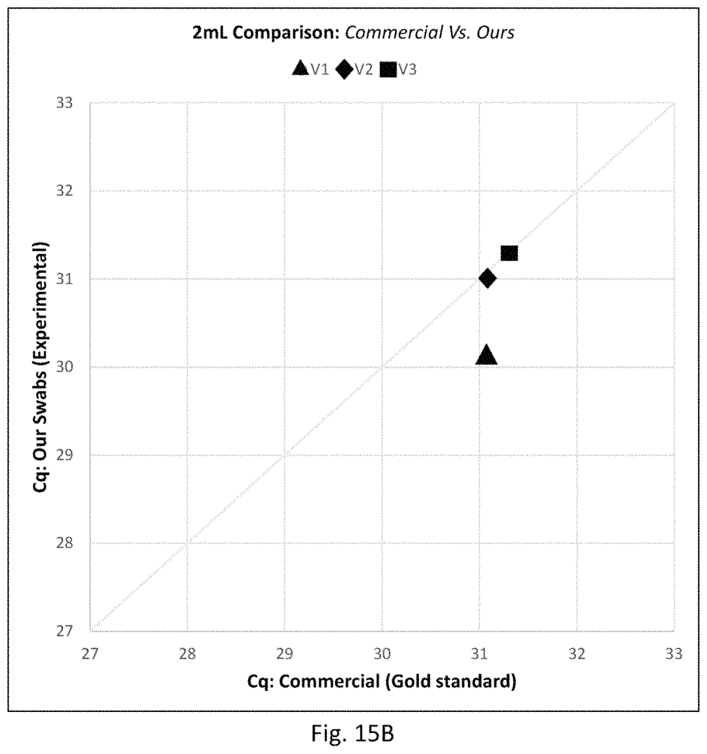

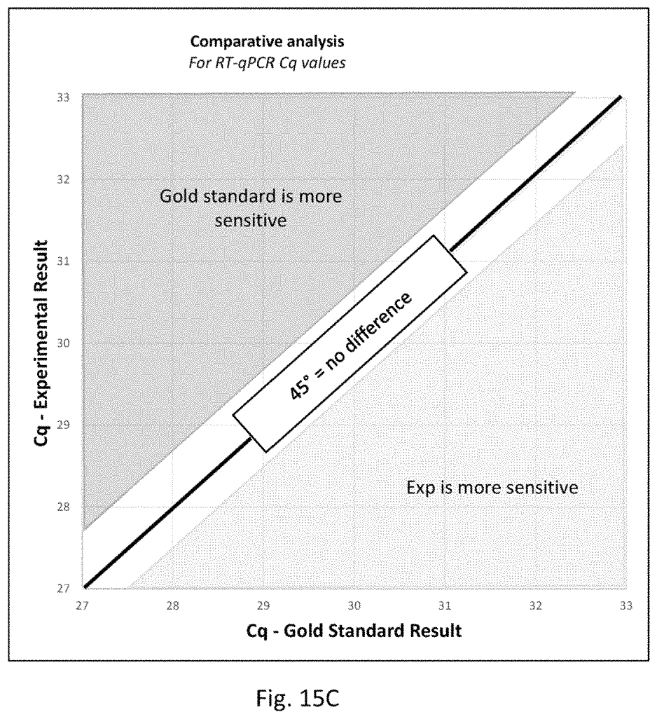

FIGS. 15A-15C illustrate various charts related to purge swabs. In particular, FIGS. 15A-15B illustrate results of comparison tests carried out using 3D printed purge swabs and comparing to a commercially-available product. FIG. 15C illustrates an interpretation key to FIGS. 15A and 15B. The axis values represent the quantitative PCR (qPCR) quantitation cycle (Cq) values measured in the lab tests. In various embodiments, a lower Cq value indicates higher sensitivity. In various embodiments, if both the experimental and standard gave the same result, then the points on the charts would fall on the 45 degree line (equal values). In various embodiments, if the standard product is more sensitive than the experimental, then the points would fall above the 45 degree line. In various embodiments, if the experimental was more sensitive than the standard, then the points would fall below the 45 degree line.

FIG. 15A illustrates the results from an experiment that investigated the effect of using a lower volume of recovery liquid (experimental volume—0.5 mL) than that generally provided with a commercial kit (standard volume—2.0 mL) to determine if the lower volume that would fit in a purge swab would negatively affect the sensitivity. As shown in FIG. 15A, all tested devices showed the lower volume being more sensitive than the commercial volume.

FIG. 15B illustrates the results from an experiment that tested both the 3D printed purge swabs (experimental—V1-3 purge swabs) with commercial swabs (standard—commercial swab) when used in the commercial recovery tubes. In the experiment, only commercial recovery tubes were used so that the only variable would be the swabs themselves. As shown in FIG. 15B, the data suggest V1 and V2 printed swabs have the same sensitivity as the commercial, but the V3 is slightly more sensitive than the commercial swab.

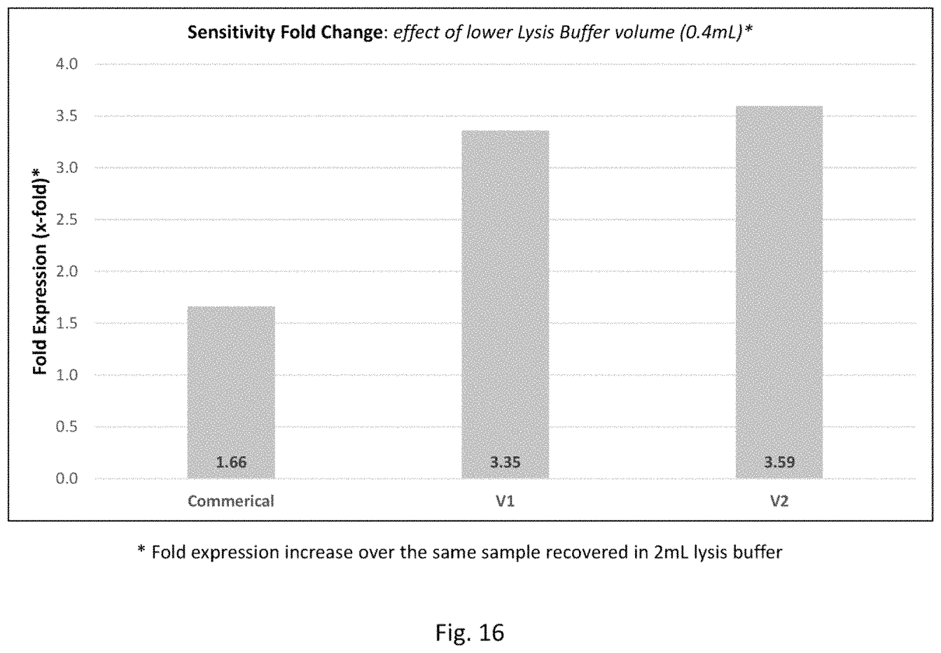

FIG. 16 illustrates a chart of sensitivity fold change. The data shown in FIG. 16 are a further analysis of sensitivity. The input data is derived from qPCR as in FIGS. 15A-15B, but the increase in sensitivity is calculated as a fold-increase over the gold standard of the commercial unit. The test was designed to examine (A) if reducing the volume of lysis buffer from the commercial 2 mL to the 0.4 mL accommodated by the purge swab would affect sensitivity, and (B) if the purge swabs (v1 and v2) would be more or less sensitive than the commercial swab in these conditions. For (A), in both the commercial swab and the purge swabs, when the volume of lysis buffer is reduced the sensitivity of the detection is increased (assuming gold standard=1.0). This result may not be surprising as the analyte is more concentrated in a smaller volume. For (B), although the commercial swab had an increase in sensitivity due to the smaller volume, the increase is higher when the purge swab (v1 and v2 swabs) are utilized. In various embodiments, this higher increase is because more material is released from the purge swab due to the purging action.

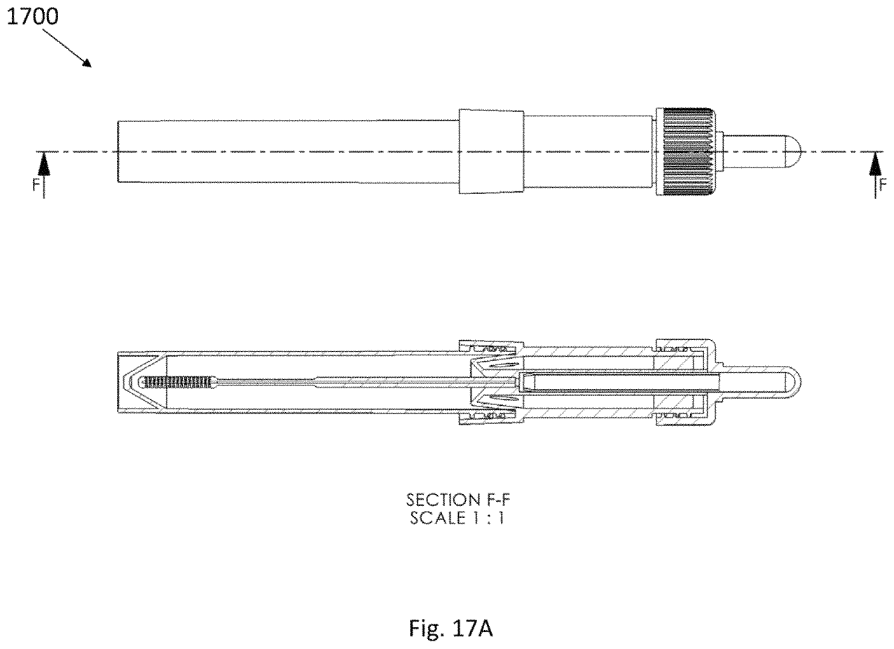

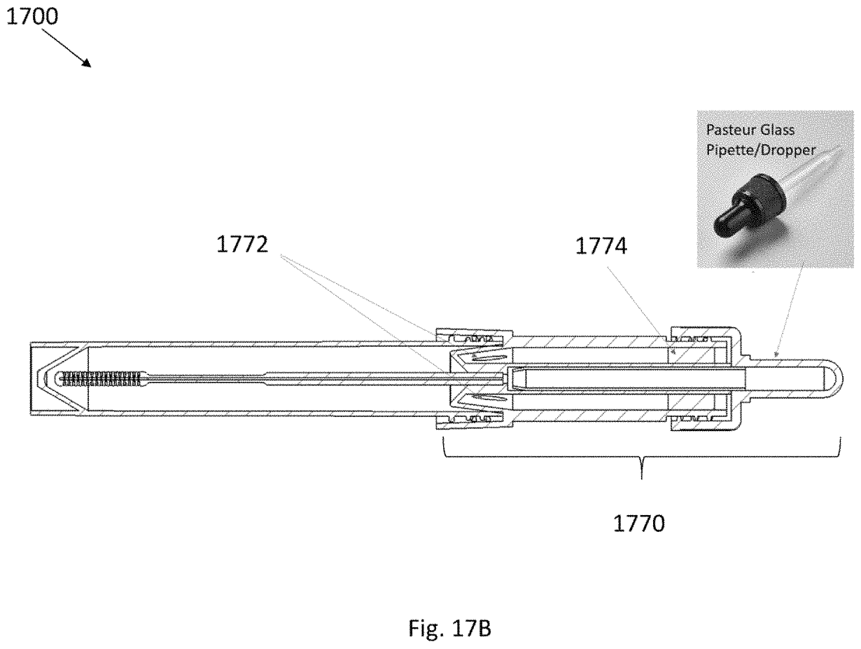

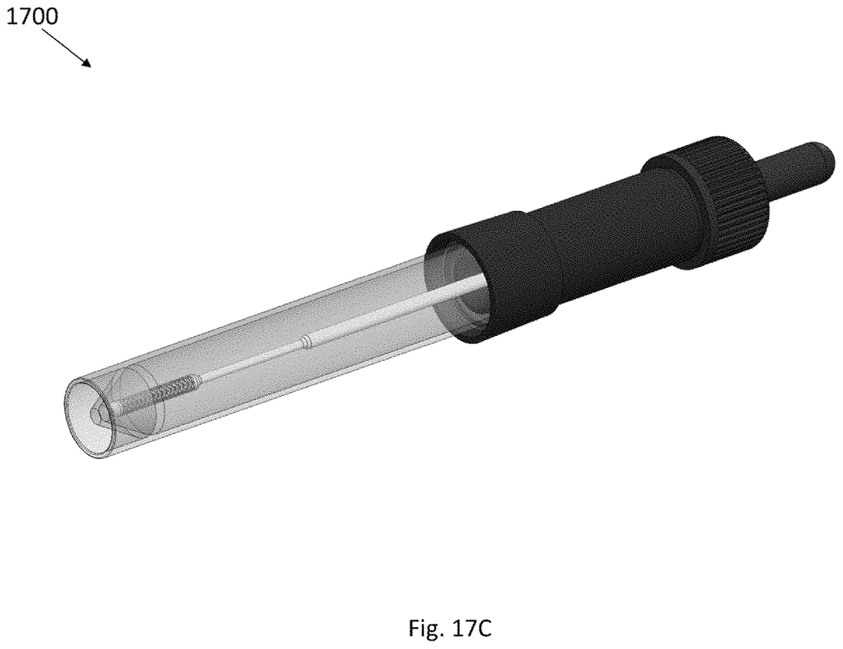

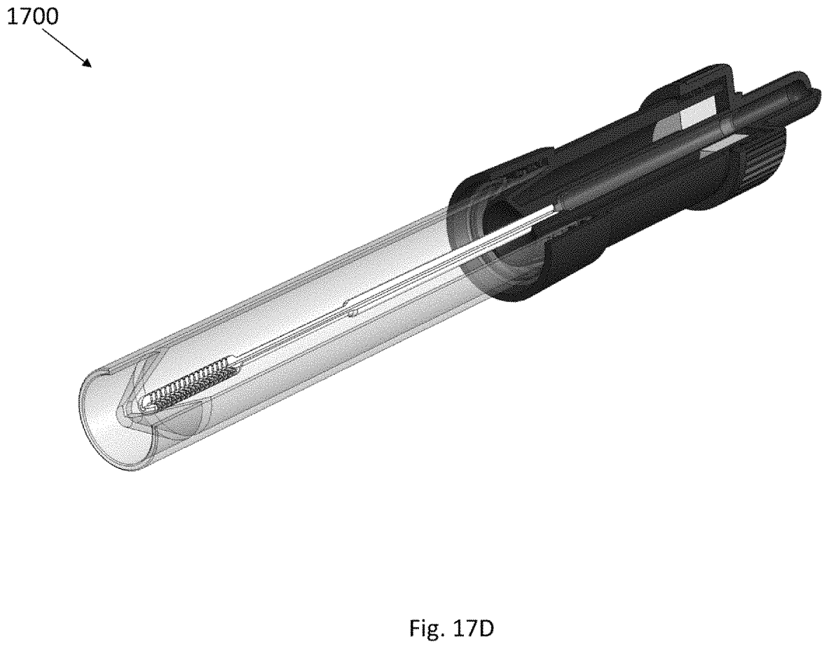

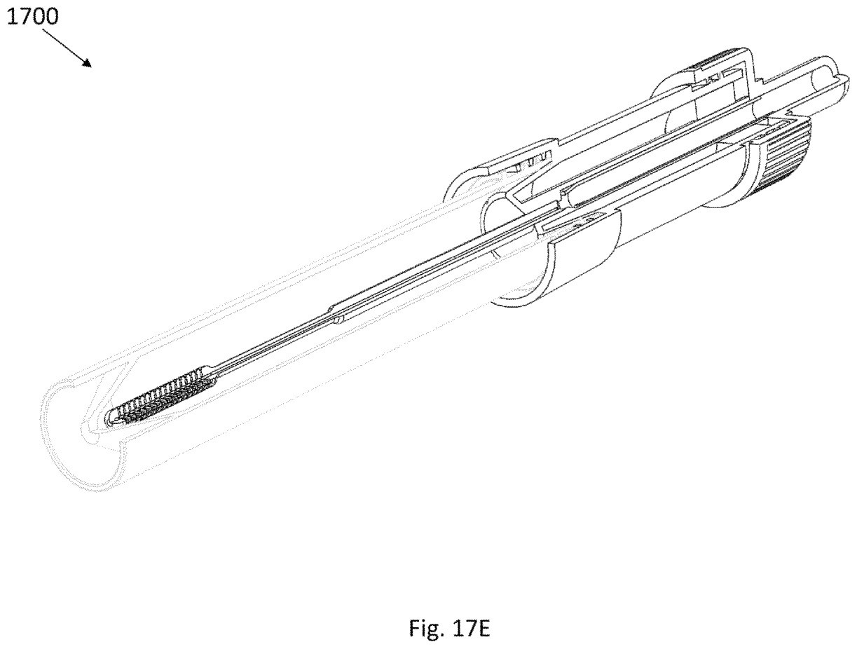

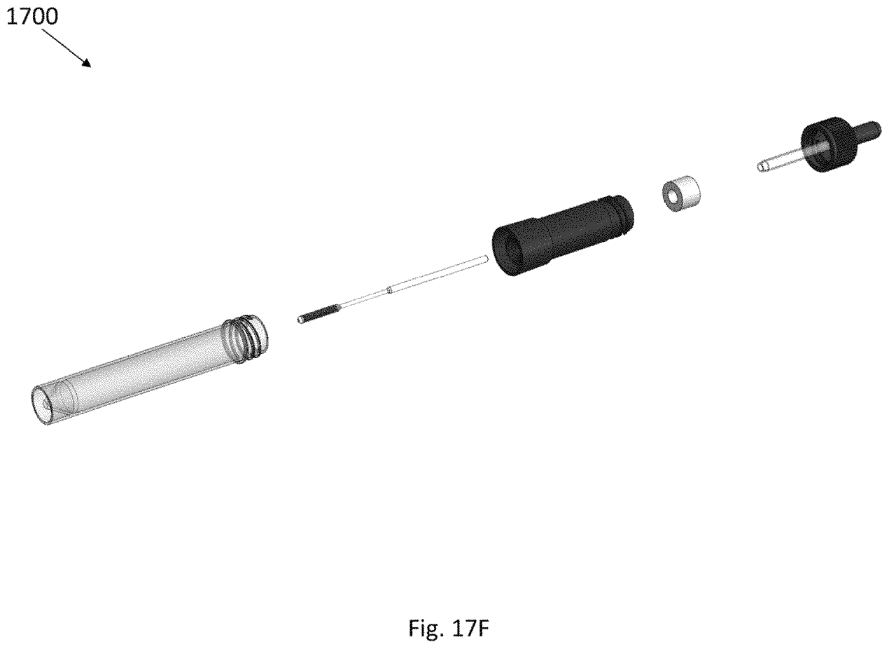

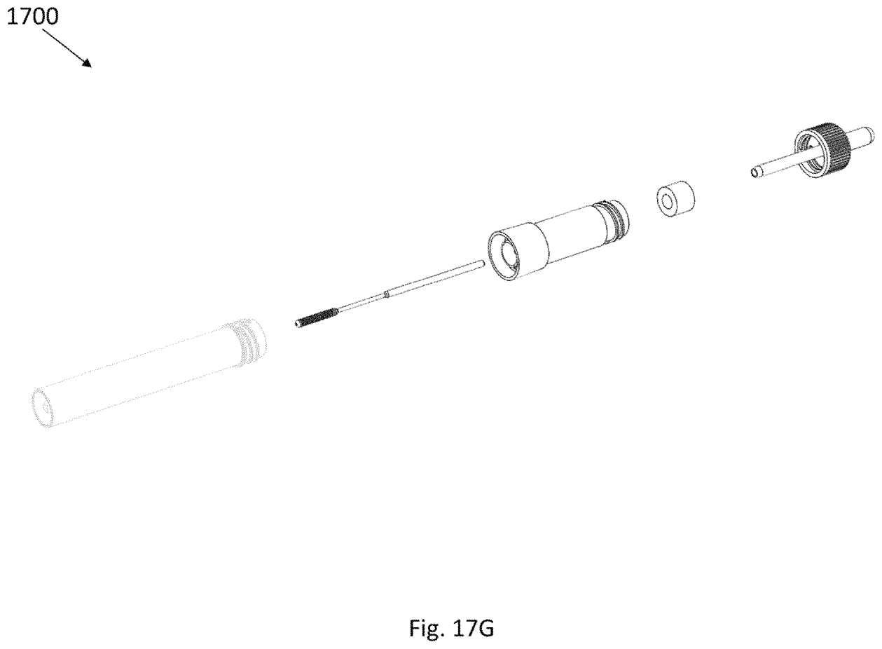

FIGS. 17A-17G illustrate a cap dropper purge swab 1700. FIG. 17A illustrates a cap dropper purge swab 1700 and a cross section view along line F-F of the cap dropper purge swab 1700. The cap dropper purge swab 1700 may be substantially similar to the purge swabs described above, but the cap of the container may be modified to incorporate a cap dropper 1770 (e.g., Pasteur pipette/dropper). FIG. 17B illustrates a cross-section view illustrating the cap dropper 1770 with one or more vents 1772 and a filter 1774, which may be similar to those commercially-available filters used in pipette tips. In various embodiments, the seating portion may extend over only a portion of the swab head. FIG. 17C illustrates a three-dimensional rendering of a cap dropper purge swab 1700. FIG. 17D illustrates a cross-sectional view of the three-dimensional rendering of a cap dropper purge swab 1700. FIG. 17E illustrates a cross-sectional view of a wire-frame rendering of a cap dropper purge swab 1700. FIGS. 17F-17G illustrate an exploded view of the cap dropper purge swab 1700.

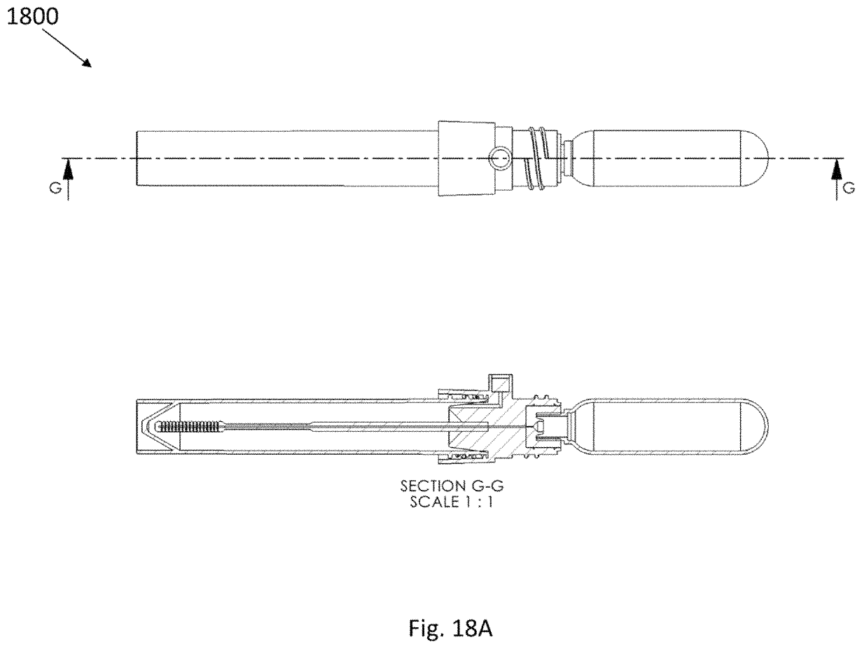

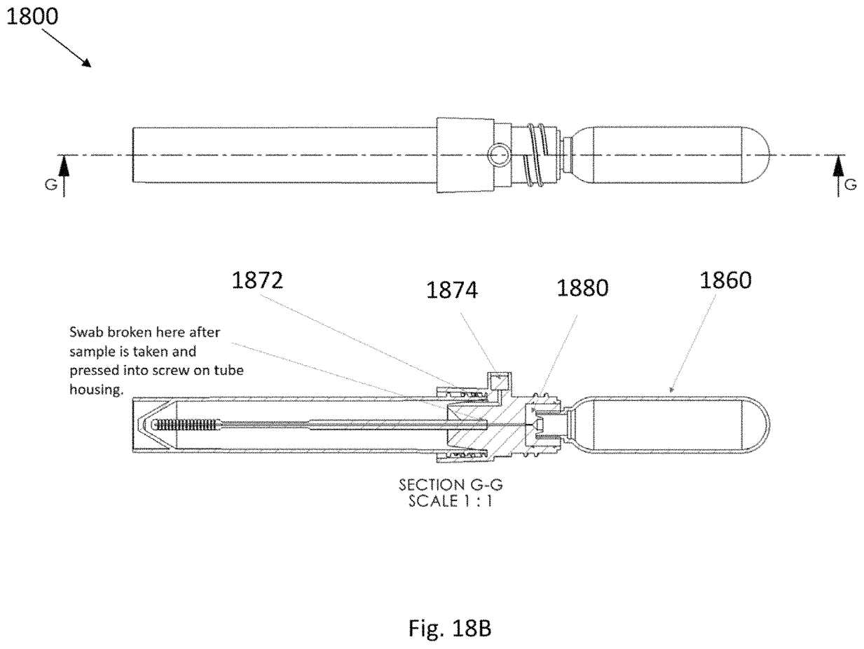

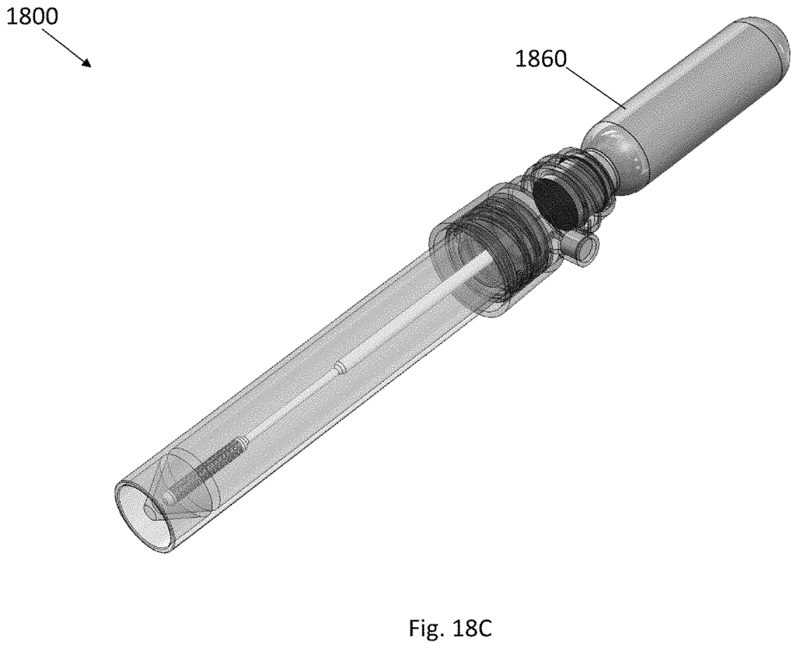

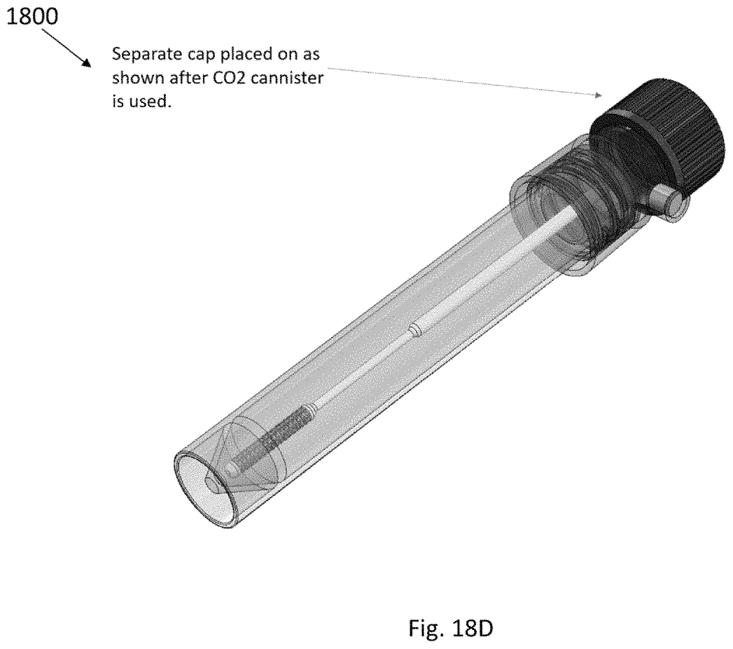

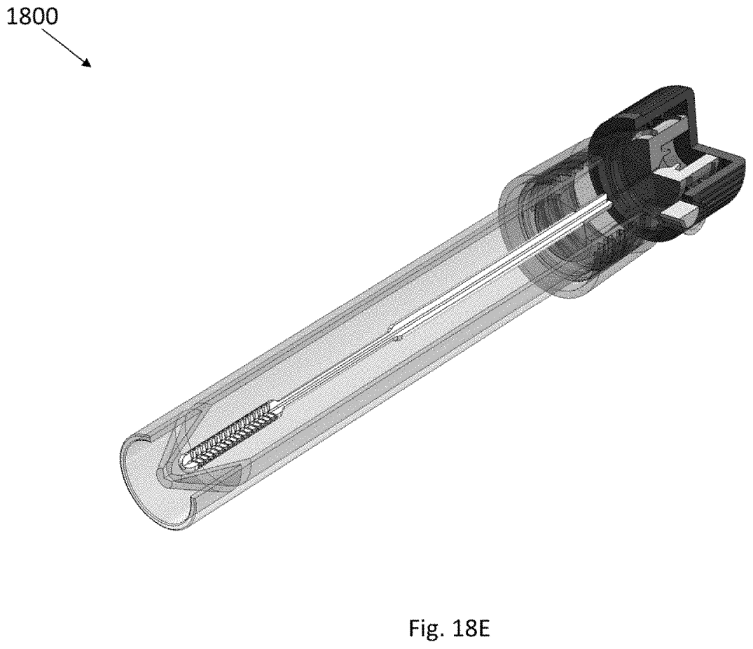

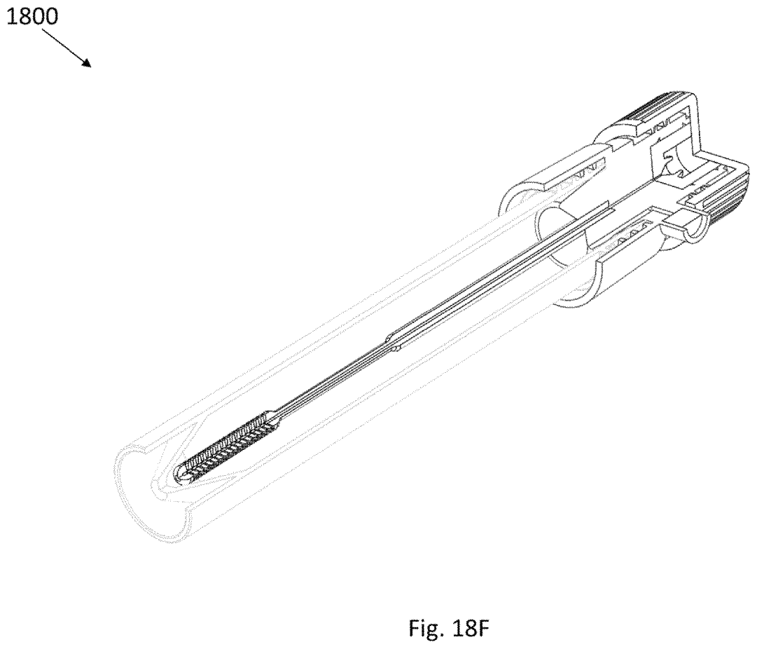

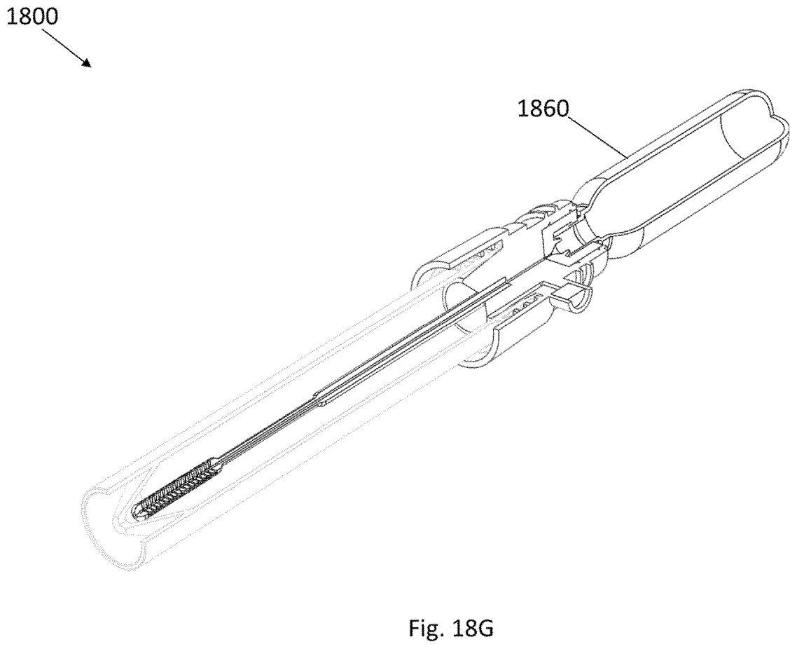

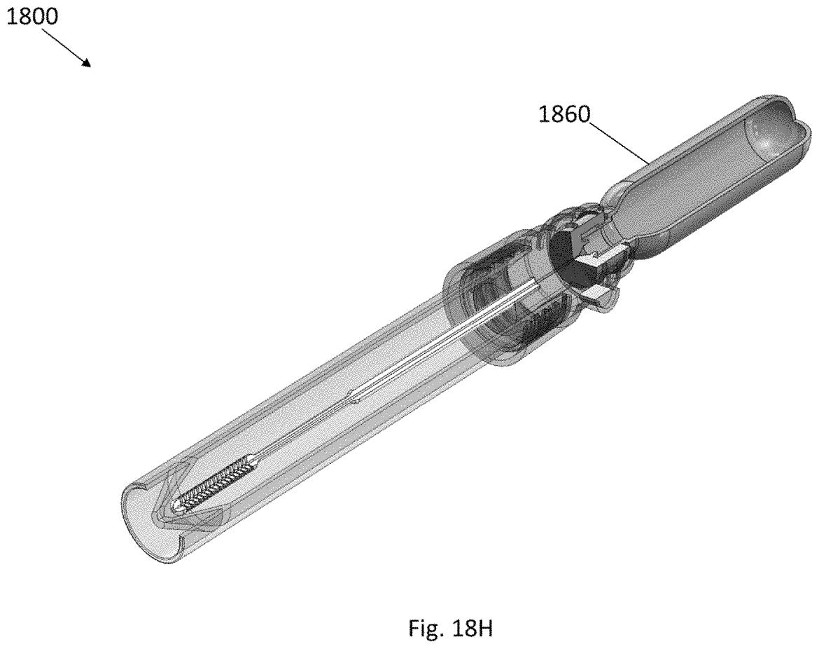

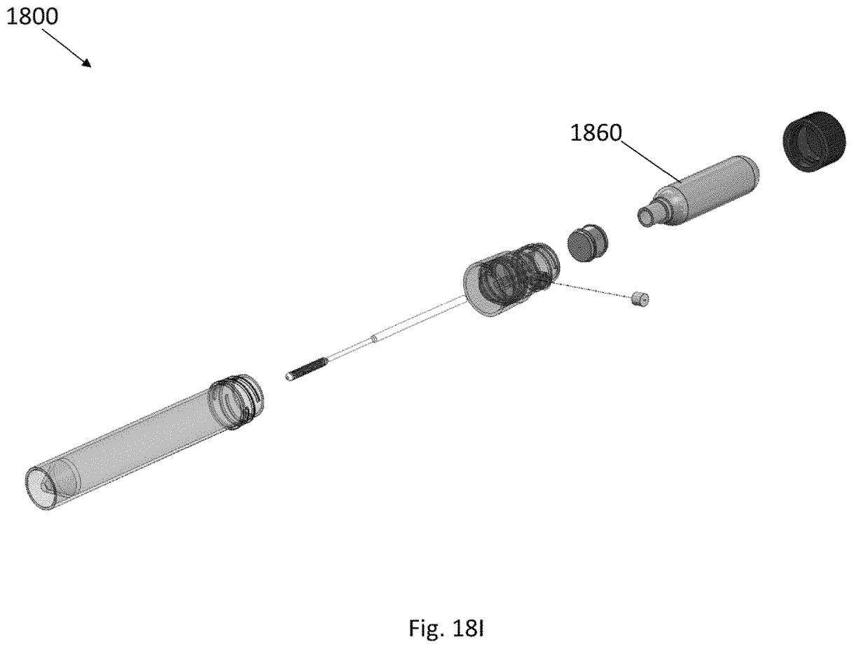

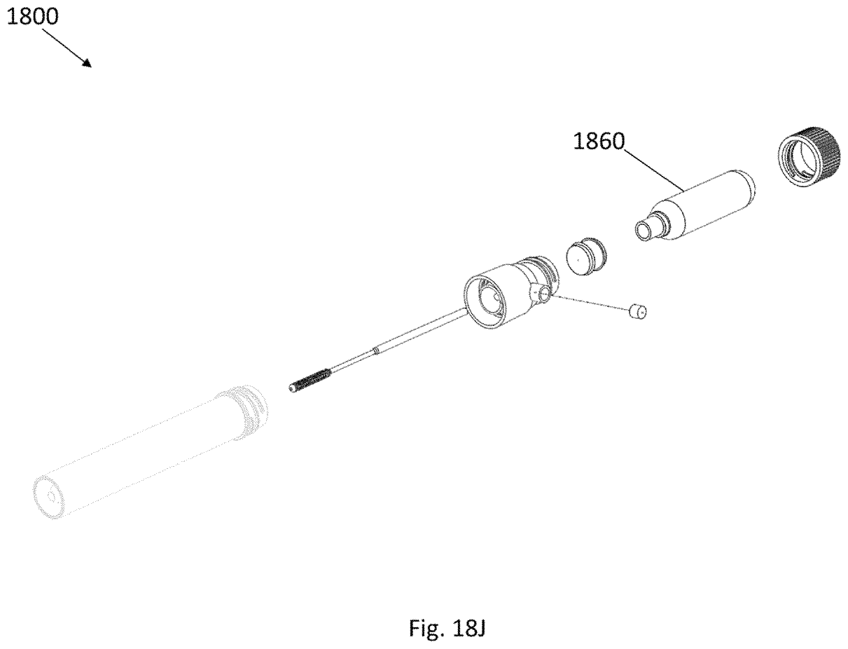

FIGS. 18A-18J illustrate a gas-powered purge swab 1800. FIG. 18A illustrates a gas-powered purge swab 1800 and a cross section view along line G-G of the gas-powered purge swab 1800. The gas-powered purge swab 1800 may be substantially similar to the purge swabs described above (e.g., FIG. 14), but the container may be modified to incorporate an insert 1880 configured to connect the compressed gas (e.g., CO₂) canister 1860. FIG. 18B illustrates a cross-section view illustrating the gas canister 1880 with one or more vents 1872 and a filter 1874, which may be similar to those commercially-available filters used in pipette tips. In various embodiments, the seating portion may extend over only a portion of the swab head. In various embodiments, the insert 1880 may include over-molded steel. In various embodiments, the insert 1880 may be configured to puncture or pierce the gas canister 1860. In various embodiments, the insert 1880 may include a metering orifice. FIG. 18C illustrates a three-dimensional rendering of a gas-powered purge swab 1800 with gas canister attached. FIG. 18D illustrates a three-dimensional rendering of a gas-powered purge swab 1800 with gas canister removed and a separate cap attached to seal the container. FIG. 18E illustrates a cross-sectional view of the three-dimensional rendering of a gas-powered purge swab 1800 with gas canister 1860 removed and a separate cap attached to seal the container. FIG. 18F illustrates a cross-sectional view of a wire-frame rendering of a gas-powered purge swab 1800 with gas canister 1860 removed and a separate cap attached to seal the container. FIG. 18G illustrates a cross-sectional view of a wire-frame rendering of a gas-powered purge swab 1800 with gas canister 1860 attached. FIG. 1811 illustrates a cross-sectional view of the three-dimensional rendering of a gas-powered purge swab 1800 with gas canister 1860 attached. FIGS. 18I-18J illustrate an exploded view of the gas-powered purge swab 1800. In various embodiments, the gas-powered purge swab 1800 may include a means to contain the purge liquid within the tube cap such that, when the gas canister 1860 is screwed on, the purge liquid will be pumped through the swab. In various embodiments, the filter 1874 is configured to prevent sample being aerosolized.

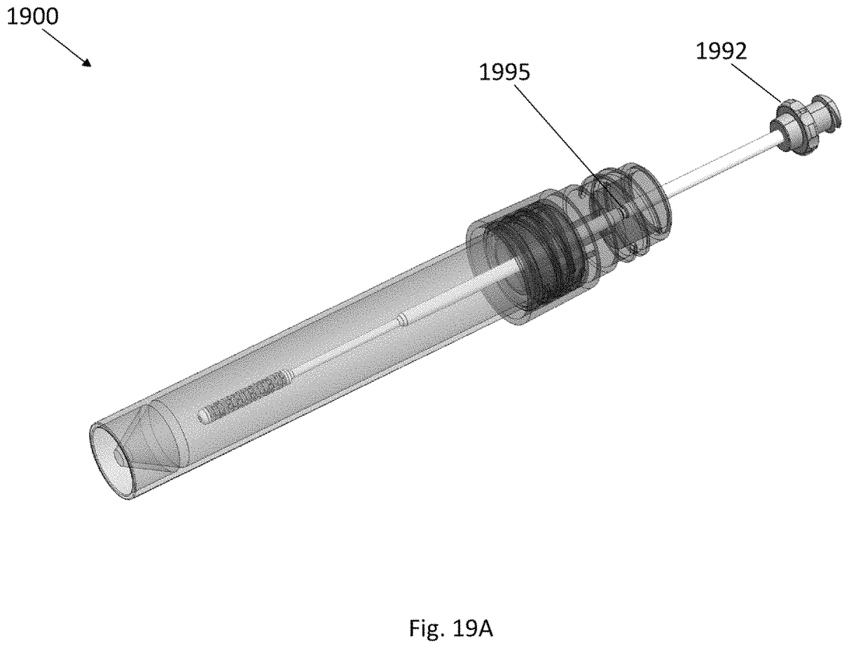

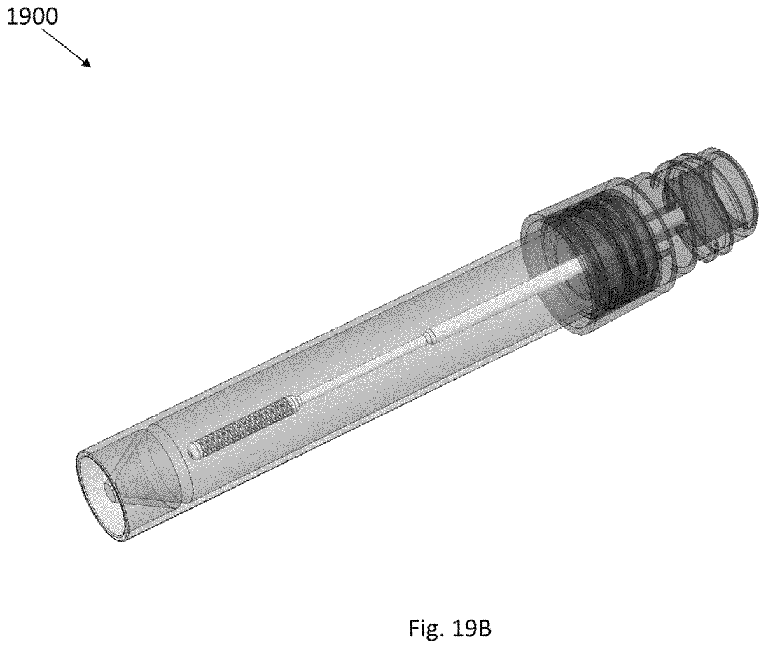

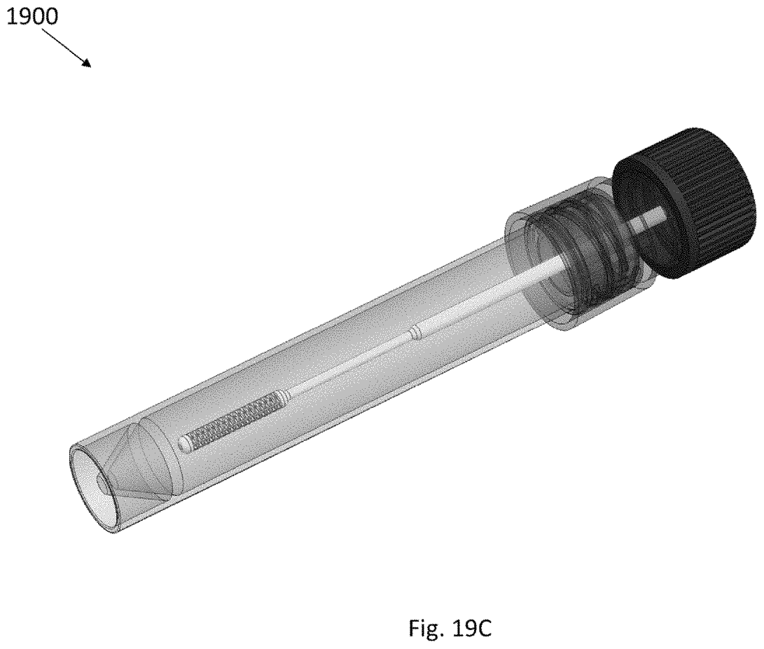

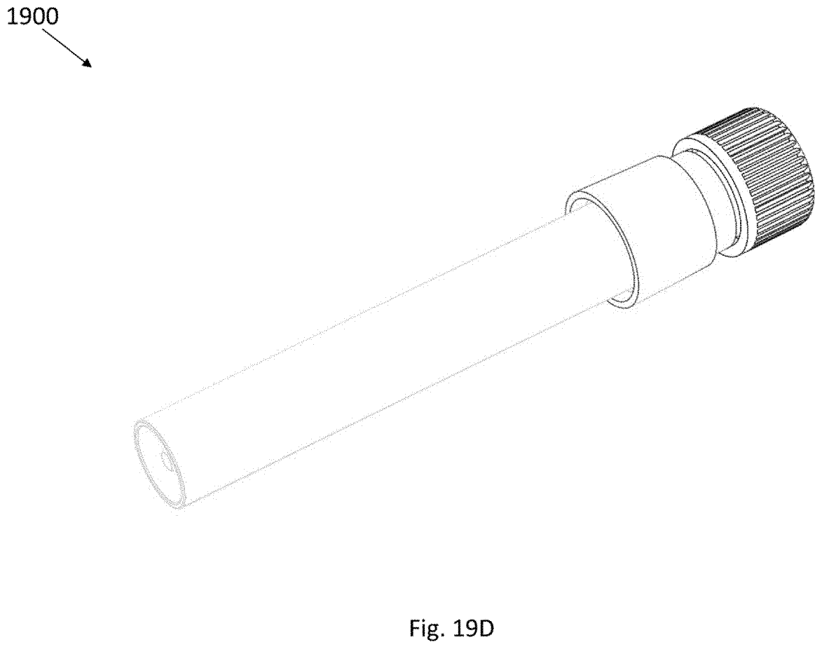

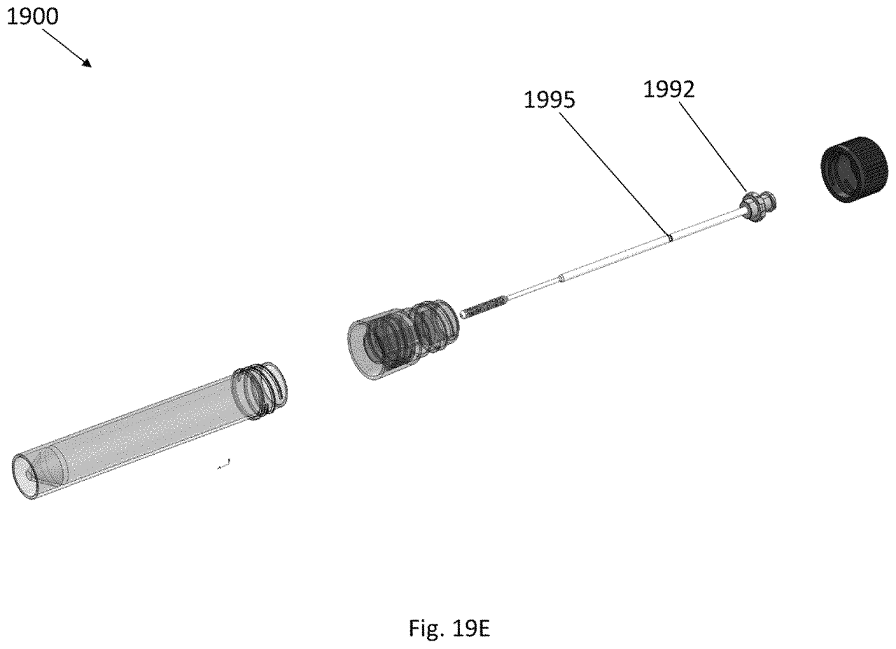

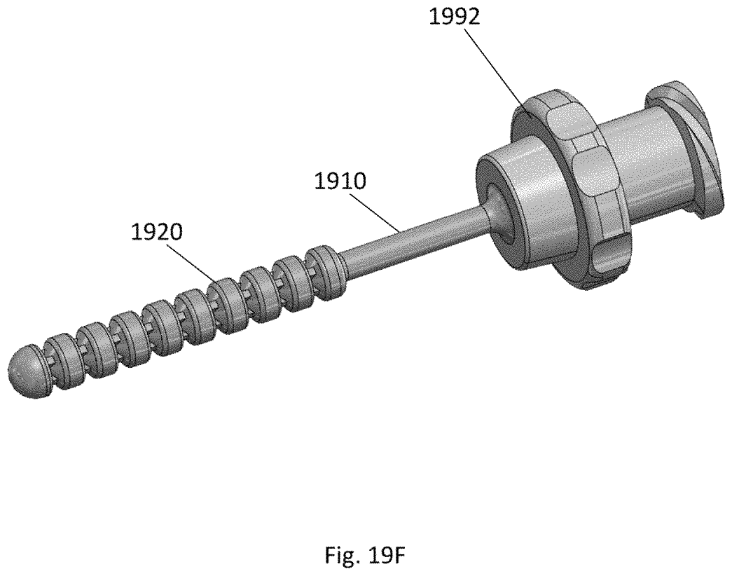

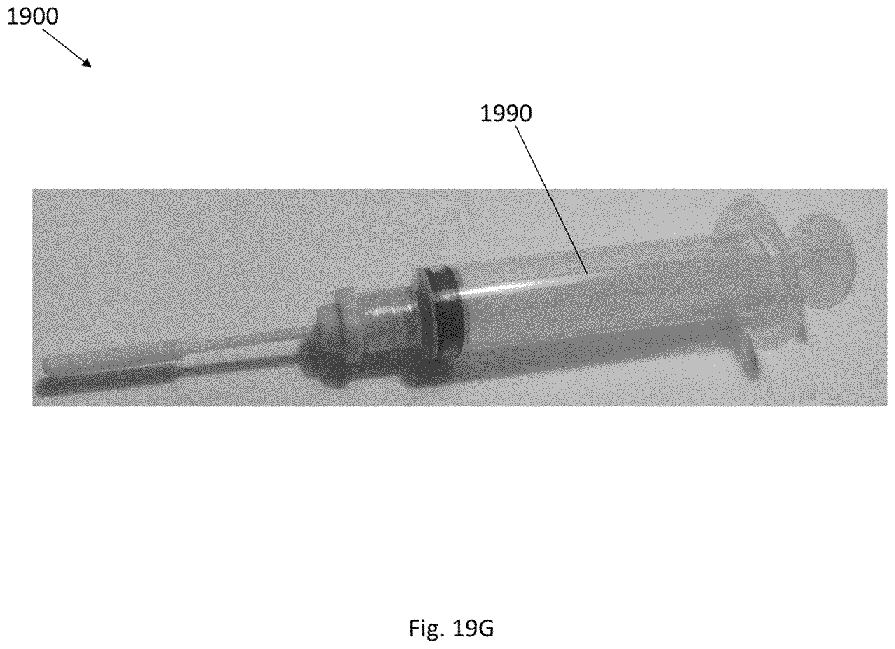

FIGS. 19A-19G illustrate a syringe purge swab according to embodiments of the present disclosure. FIG. 19A illustrates a three-dimensional rendering of a syringe purge swab 1900 configured with a full length purge swab (with a luer attachment 1992 for a syringe 1990) placed into the sample tube through a cap. In various embodiments, a syringe 1990 is then attached to the luer attachment 1992 to flush the purge liquid through the swab head and into the container. In various embodiments, the swab is broken off at the fracture site after purging. In various embodiments, after the swab is broken off, the container is capped with a separate cap. In various embodiments, the syringe purge swab 1900 may be substantially similar to the purge swabs described above. FIG. 19B illustrates a three-dimensional rendering of a syringe purge swab 1900 after the swab has been broken at a frangible region 1995 but before the container is capped. FIG. 19C illustrates a three-dimensional rendering of a syringe purge swab 1900 after the swab has been broken at a frangible region 1995 and after the container is capped. FIG. 19D illustrates a wire-frame rendering of a syringe purge swab 1900 after the swab has been broken at a frangible region 1995 and after the container is capped. FIG. 19E illustrate an exploded view of the syringe purge swab 1900. FIG. 19F illustrates a shorter version of the syringe purge swab where a luer attachment 1992 is directly attached to a purge swab body 1910 having a swab head 1920. In various embodiments, this syringe purge swab may have applications for irrigation during surgeries. In various embodiments, as shown in FIG. 19G, the syringe 1990 is attached and used to handle the swab during the rinse/irrigation procedure.

While the disclosed subject matter is described herein in terms of certain preferred embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

What is claimed is:

1. An apparatus comprising:
an elongated body having a proximal end, a distal end, and a length therebetween;
a swab head having a proximal end and a distal end, the proximal end of the swab head attached to the distal end of the elongated body, wherein the swab head comprises a plurality of openings; and
a lumen extending from the proximal end of the elongated body into at least a portion of the swab head, wherein the lumen is in fluidic communication with the plurality of openings.

2. The apparatus of claim 1, wherein the swab head comprises a plurality of radial extrusions, the radial extrusions extending from the lumen to an outer circumference of the swab head.

3. The apparatus of claim 1, wherein the swab head comprises a plurality of concentric rings, each concentric ring extending from the lumen to an outer circumference of the swab head.

4. The apparatus of claim 3, wherein each of the plurality of concentric rings is scored on the outer circumference.

5. The apparatus of claim 1, wherein the lumen is configured to receive a fluid at the proximal end of the elongated body such that the fluid flows through the lumen and out the plurality of openings.

6. The apparatus of claim 1, further comprising a sample collection material disposed on the swab head.

7. A kit comprising:
the apparatus of claim 1; and
a container having a proximal end and a distal end, wherein the proximal end is configured to receive the apparatus, wherein the distal end comprises a seating portion having a smaller diameter than a diameter of the proximal end.

8. The kit of claim 7, further comprising a source of pressurized gas configured to couple to the cap to thereby apply the pressurized gas to the lumen.

9. An apparatus comprising:
an elongated body having a proximal end, a distal end, and a length therebetween along a longitudinal axis;
a swab head having a proximal end and a distal end, the proximal end of the swab head attached to the distal end of the elongated body, wherein the swab head comprises a plurality of openings,
a bore extending along the longitudinal axis from the distal end of the swab head at least partially into the swab head;
wherein the bore is in fluidic communication with the plurality of openings, wherein the bore is configured to engage a pin of a container when the swab is inserted into the container thereby forcing fluid through the plurality of openings.

10. The apparatus of claim 9, wherein the swab head comprises a plurality of grooves on an outer surface of the swab head.

11. The apparatus of claim 10, wherein the plurality of grooves comprise a helical groove.

12. The apparatus of claim 9, further comprising a sample collection material disposed on the swab head.

13. A kit comprising:
the apparatus of claim 9; and
a container having a proximal end and a distal end, wherein the proximal end is configured to receive the apparatus, wherein the distal end comprises a seating portion, the seating portion having a smaller diameter than a diameter of the proximal end and a pin extending from the distal end along the longitudinal axis.

14. The kit of claim 13, wherein the container comprises a plurality of fins extending radially inward from an inner surface of the container.

15. An apparatus comprising:
an elongated body having a proximal end, a distal end, and a length therebetween;
a swab head having a proximal end and a distal end, the proximal end of the swab head attached to the distal end of the elongated body;
wherein the swab head comprises a plurality of surface features on an outer surface of the swab head.

16. The apparatus of claim 15, wherein the swab head does not include any openings.

17. The apparatus of claim 15, wherein the plurality of surface features comprises grooves.

18. The apparatus of claim 17, wherein the grooves are helical grooves.

19. The apparatus of claim 15, wherein the plurality of surface features comprises bumps.

20. The apparatus of claim 15, wherein the plurality of surface features comprises scoring.

21. A kit comprising:
the apparatus of claim 15; and
a container having a proximal end and a distal end, wherein the proximal end is configured to receive the apparatus, wherein the distal end comprises a seating portion having a smaller diameter than a diameter of the proximal end, wherein the seating portion comprises one or more engagement structure.

22. The kit of claim 21, wherein the one or more engagement structure comprises a helical groove.

23. The kit of claim 21, wherein the one or more engagement structure comprises a helical rib.

24. The kit of claim 21, wherein the one or more engagement structure comprises a plurality of circumferential ribs.

25. The kit of claim 21, wherein the one or more engagement structure comprises a plurality of circumferential grooves.

26. A method comprising:
providing a kit of claim 7;
contacting the swab head against a biological sample to thereby obtain at least a portion of the biological sample on the swab head;
inserting the swab into the container; and
affixing a cap on the proximal end of the container such that the cap couples to the lumen in the elongated body to thereby force a fluid through the elongated body and out of the plurality of openings in the swab head.

27. The method of claim 26, further comprising removing a portion of the proximal end of the elongated body.

28. A method comprising:
providing a kit of claim 7;
contacting the swab head against a biological sample to thereby obtain at least a portion of the biological sample on the swab head;
inserting the swab into the container;
affixing a cap on the proximal end of the container such that the cap couples to the lumen in the elongated body and substantially seals the apparatus in the container; and
applying a pressurized gas through the cap to the lumen to thereby force a fluid through the elongated body and out of the plurality of openings in the swab head.

29. A method comprising:
providing a kit of claim 13;
contacting the swab head against a biological sample to thereby obtain at least a portion of the biological sample on the swab head;
inserting the swab into the container such that the bore of the swab head receives the pin; and
affixing a cap on the proximal end of the container such that the cap substantially seals the apparatus in the container.

30. A method comprising:
providing a kit of claim 21;
contacting the swab head against a biological sample to thereby obtain at least a portion of the biological sample on the swab head;
inserting the swab head into the container such that the swab head engages the one or more engagement structure;
affixing a cap on the proximal end of the container such that the cap substantially seals the apparatus in the container.
